(19) United States
(12) Patent Application Publication
(10) Pub. No.: US 2009/0269746 A1
(43) Pub. Date: Oct. 29, 2009

(54) MICROSEQUENCER-WHOLE GENOME SEQUENCER

(76) Inventors: Gil Atzmon, Bronx, NY (US); Igal Chertkow, Ashdod (IL); Temuri Budagov, Elmwood Park, NJ (US)

Correspondence Address: BURNS & LEVINSON, LLP, 125 SUMMER STREET, BOSTON, MA, 02110, US

(21) Appl. No.: 12/109,934

(22) Filed: Apr. 25, 2008

(51) Int. Cl. C12Q 1/68; C12M 1/34

(52) U.S. Cl. 435/6; 435/287.2

(57) ABSTRACT

The method and apparatus are disclosed to support speedy sequencing of genomes of individuals. The method comprises random digestion of a stretch of DNA; adaptor ligation of adaptor DNA fragments to DNA segments produced in random digestion, each said adaptor DNA fragment containing a sequence which is complementary to a single DNA primer; PCR amplification of the ligated segments produced in adaptor ligation, utilizing a single DNA primer; distributing the ligated segments into one or more pre-defined isolated locations of a sequencing apparatus, each said location containing DNA fragments placed there for capturing a unique kind of digested DNA segments; capturing at each location a unique kind of amplified DNA segments by hybridization with the DNA fragments, dislodging captured DNA segments from DNA fragments; adding DNA sequencing reaction components into the locations; performing sequencing reactions at each location; separating the products of the sequencing reactions in the sequencing apparatus; and determining the sequences of DNA segments captured at individual locations of the sequencing apparatus. The apparatus comprises one or more isolated locations, each location has a reservoir containing DNA fragments placed there for capturing a unique kind of DNA segments from a DNA solution after dispensing the DNA solution into the reservoir; one or more channels performing DNA separation according to size, said channels being associated with one or more reservoirs; one or more gates controlling the flow of substances in the reservoirs; an optical system which induces fluorescence excitation in, and detects fluorescence emission in the channels; and a computer to produce DNA sequence data.

### FIELD OF THE INVENTION

The present invention is related generally to the field of genome sequencing. More particularly, the present invention is related to a highly cost and time efficient process utilizing a novel apparatus for parallel sequencing of a genome of an individual representative of a species.

### BACKGROUND OF THE INVENTION

The primary goal of the Human Genome Project (HGP) was to increase the throughput of genome sequencing while reducing sequencing cost. Solving this problem helped to accelerate the completion of the HGP. At the point of the HGP completion the sequencing capacity was far greater than at the inception of the HGP. Achieving project sequencing goals required a two- to threefold improvement. Incremental advances in sequencing technologies, efficiency, and cost were accomplished. DNA sequencing costs have fallen significantly, fueled in large part by tools, technologies and process improvements developed as part of the successful effort to sequence the human genome. However, sequencing of three billion base pairs—the amount of DNA found in the genomes of humans and other mammals, still comes at a significant cost and time.

### SUMMARY OF THE INVENTION

It is an aim of this invention to reduce the cost and the time that is required to sequence a whole genome of an individual representative of a species and to enable determining genome sequences for individual representatives of every species for which genome sequence data is available, which includes humans. A method and an apparatus are suggested herein that enable multiple simultaneous sequencing of genome sequences for individuals at a low cost per genome in a matter of hours.

While the primary focus of this invention is parallel sequencing of genomes, it should be obvious to one skilled in the art that the concepts disclosed herein may be adapted for other purposes by fully or partially utilizing equivalent steps, methods and devices. Such other purposes include, but are not limited to, parallel detection of small amounts of individual components in mixtures of various substances, such as hazards, chemicals or pollutants, by utilizing various kinds of specific molecular probes. In this sense, while the focus of this disclosure is on DNA it is broadly understood that DNA can be replaced with any other substance that is intended to be monitored, detected, analyzed, separated or treated in any possible way. The same is true for mnemonics such as fragments, oligonucleotides, ligands, etc. that those skilled in the art may find to be adequate replacements for each specific application,

### BRIEF DESCRIPTION OF THE DRAWINGS

The present invention is illustratively shown and described in reference to the accompanying drawings, in which

FIG. 1a is a block diagram illustrating the general concept of breaking down a whole genome into fragments that are amenable to parallel sequencing;

FIG. 1b is a block diagram illustrating the general concept of preparing the fragments of the whole genome for parallel sequencing;

FIG. 1c is a block diagram illustrating the general concept of parallel sequencing of the whole genome fragments and of reconstructing the whole genome DNA map;

FIG. 2a is an illustration of the whole genome digestion into random fragments, followed by adaptor ligation and PCR amplification of the ligated fragments;

FIG. 2b illustrates the concept of an array of sequencing locations, the details of an individual location are shown in the insets;

FIG. 2c further illustrates the concept of an array of sequencing locations, each having an individual DNA sequence immobilized therein;

FIG. 2d illustrates an individual sequencing location from the array with a corresponding DNA separation channel;

FIG. 2e illustrates the separation of DNA fragments in a single channel of the array, and DNA fluorescence excitation, emission, detection and processing;

FIG. 3a illustrates a DNA sequencing apparatus embodiment with a DNA holding structure and a “ball” type gate structure at each sequencing reaction location;

FIG. 3b illustrates hybridization of the amplified genomic DNA fragments with the DNA immobilized on the holding structure of the sequencing apparatus;

FIG. 3c illustrates addition of the sequencing reaction components into the sequencing locations of the apparatus;

FIG. 3d illustrates sealing of the sequencing locations of the apparatus and dislodging of captured genomic DNA fragments;

FIG. 3e illustrates the contents and configuration of the sequencing locations after lifting the DNA holding structure;

FIG. 3f illustrates the configuration of the sequencing locations during the sequencing PCR;

FIG. 3g illustrates DNA separation by electrophoresis in the channels associated with the sequencing locations;

FIG. 3h illustrates fluorescence excitation and emission from the DNA fragments separated in the channels of the sequencing apparatus, and collection of DNA fragments exiting the channels;

FIG. 3i illustrates the apparatus embodiment which has no gates, and with a DNA holding structure in its open position;

FIG. 3j illustrates the apparatus embodiment which has no gates, and with a DNA holding structure in its closed position;

FIG. 4 is a schematic illustration of the details of a rotating gate action;

FIG. 5a is a schematic illustration of electric and/or magnetic force actuation of a rotating gate;

FIG. 5b is a schematic illustration of electric and/or magnetic force actuation of a translating gate;

FIG. 5c is a schematic illustration of electric and/or magnetic force actuation of a flexing gate;

FIG. 5d is a schematic illustration of electric and/or magnetic force actuation of a flexing gate with SCRATCH drive actuators;

FIG. 6a is a schematic illustration of a closed position of a gate structure that controls adjacent sequencing locations;

FIG. 6b is a schematic illustration of an open position of a gate structure that controls adjacent sequencing locations;

FIG. 7 is a pictorial illustration of an example of a rotating gate fabrication utilizing MEMS technology;

FIG. 8a a is a schematic illustration of a non-actuated configuration of a “ball” type gate structure actuated through a combined action of two force domains;

FIG. 8b is a schematic illustration of an actuated configuration of a “ball” type gate structure with charged electrodes shown to repel similar charged proximal “balls;”

FIG. 8c is a schematic illustration of an actuated configuration of a “ball” type gate structure with swapped “ball” positions and charged electrodes shown to attract similar charged proximal “balls;”

FIG. 8d is a schematic illustration of a “ball” type gate structure with additional electrodes to promote unobstructed motion of the “balls” during actuation;

FIG. 9 schematically illustrates various possible locations for oligonucleotide immobilization at a sequencing location;

FIG. 10 is a schematic illustration of the optical detection system;

FIG. 11 is a schematic illustration of a curved channel configuration implemented in a single substrate plate; and

FIG. 12 is a schematic illustration of configuring channels utilizing multiple substrate plates.

### DETAILED DESCRIPTION OF THE INVENTION

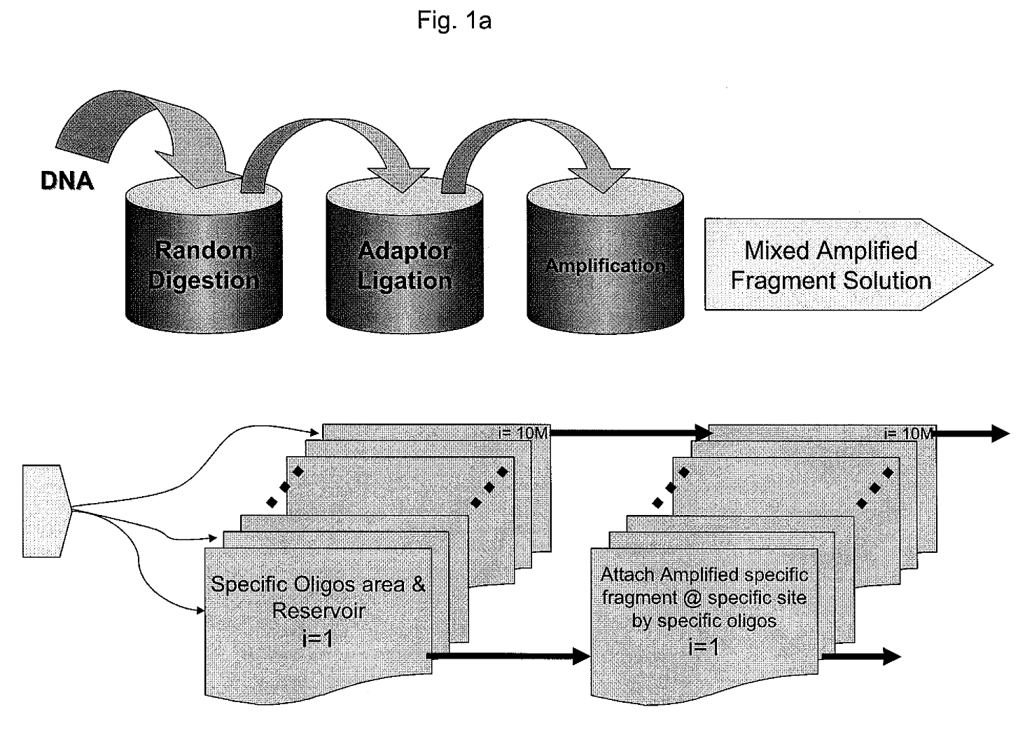

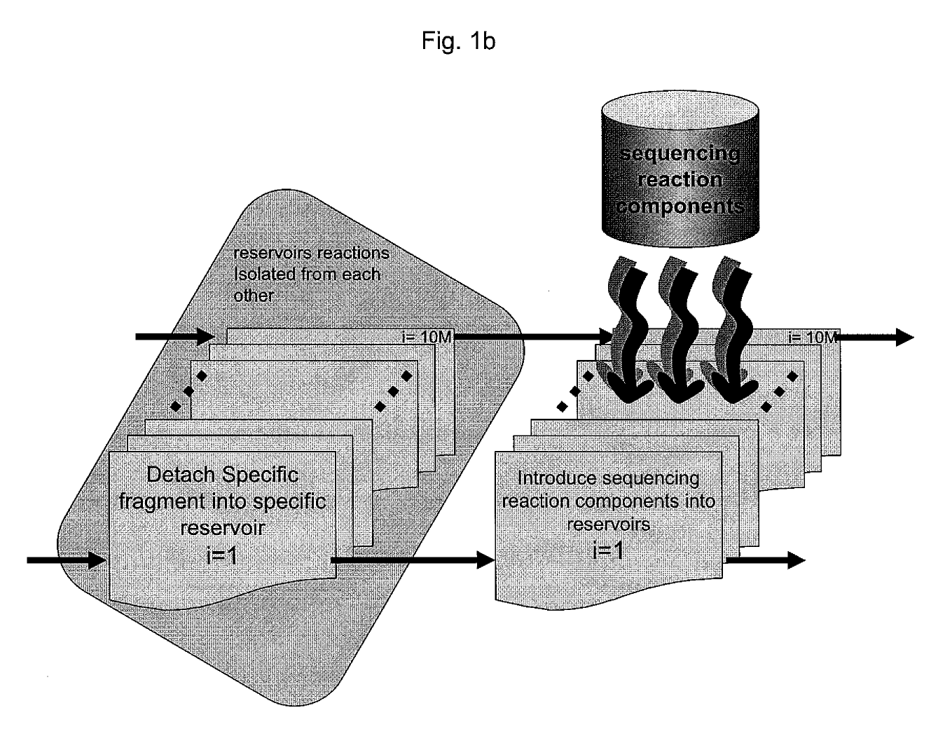

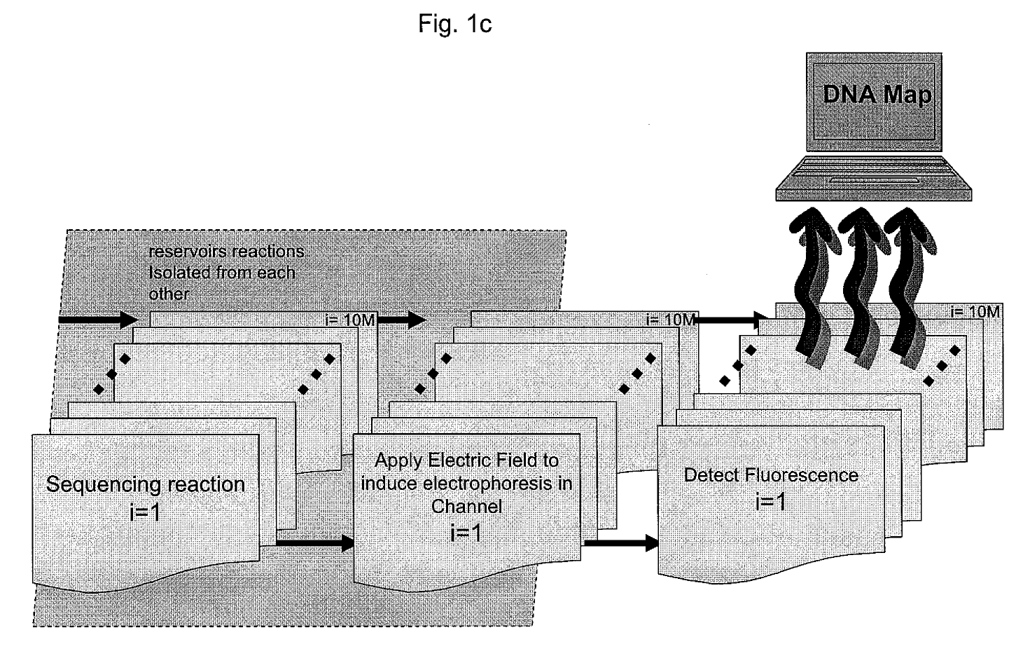

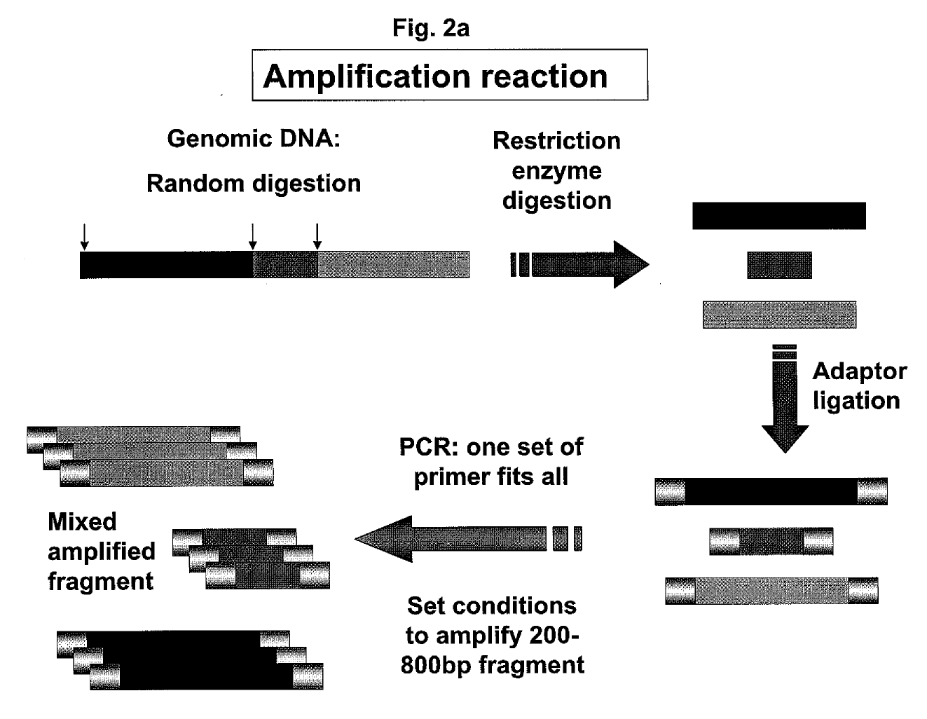

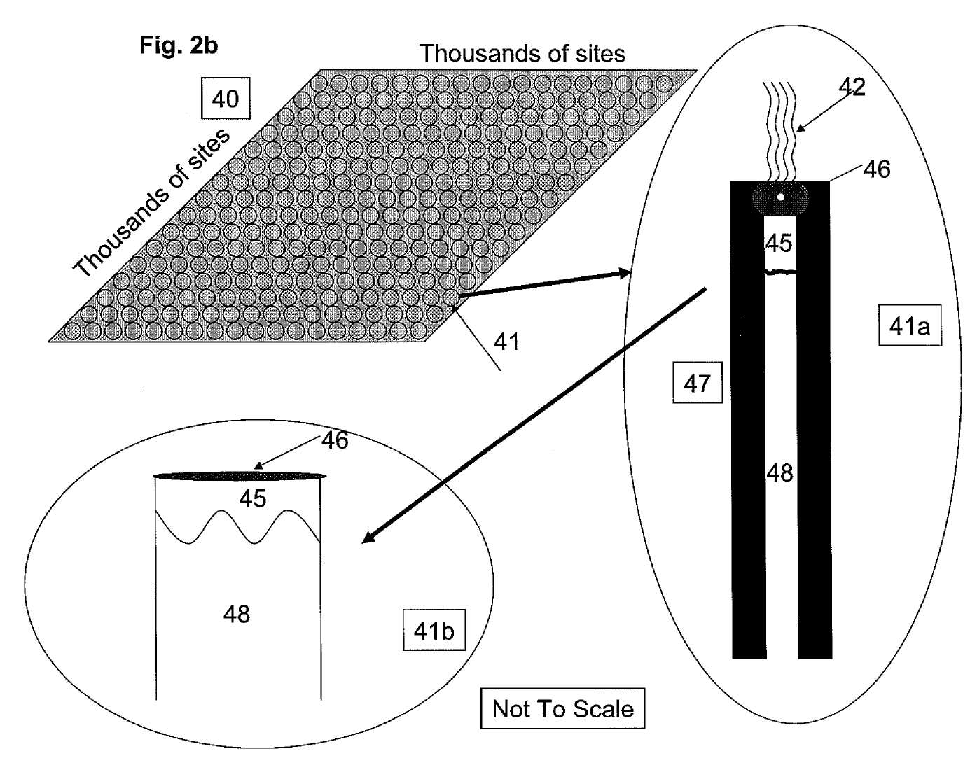

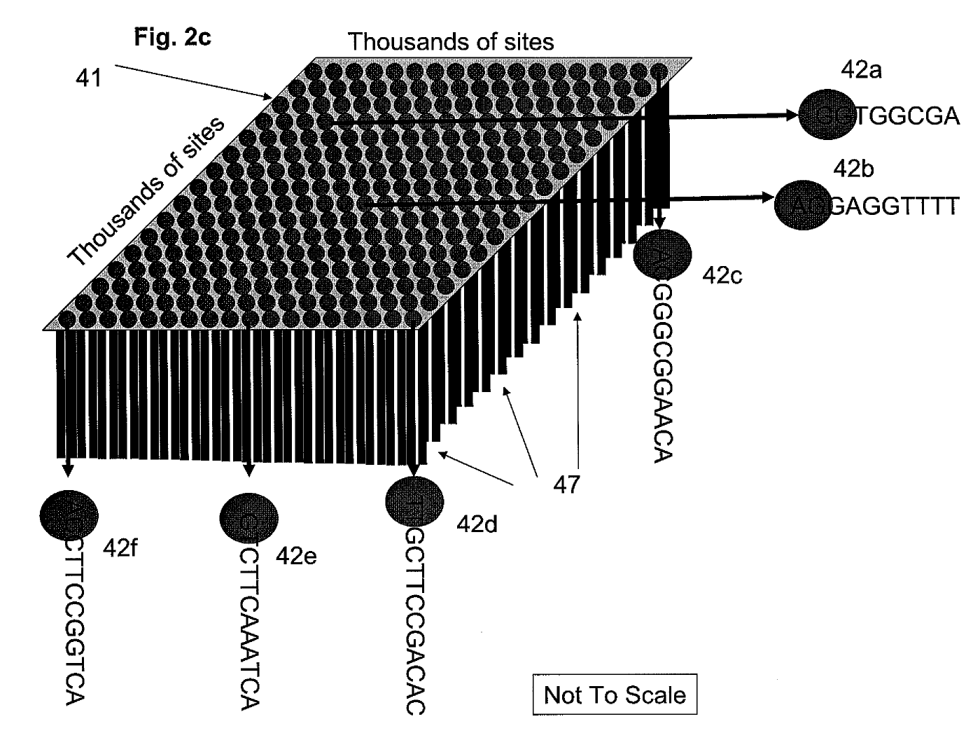

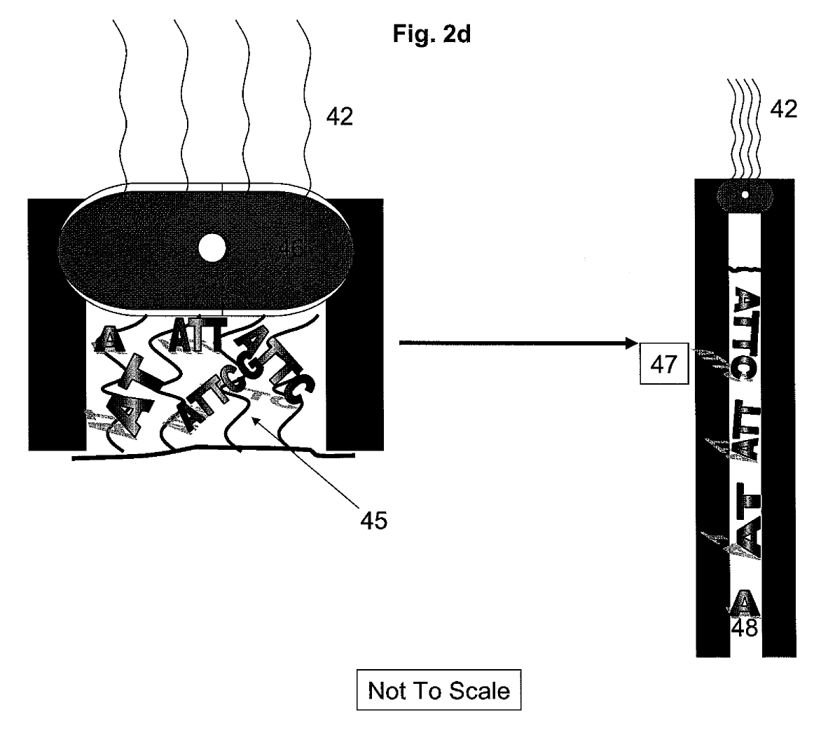

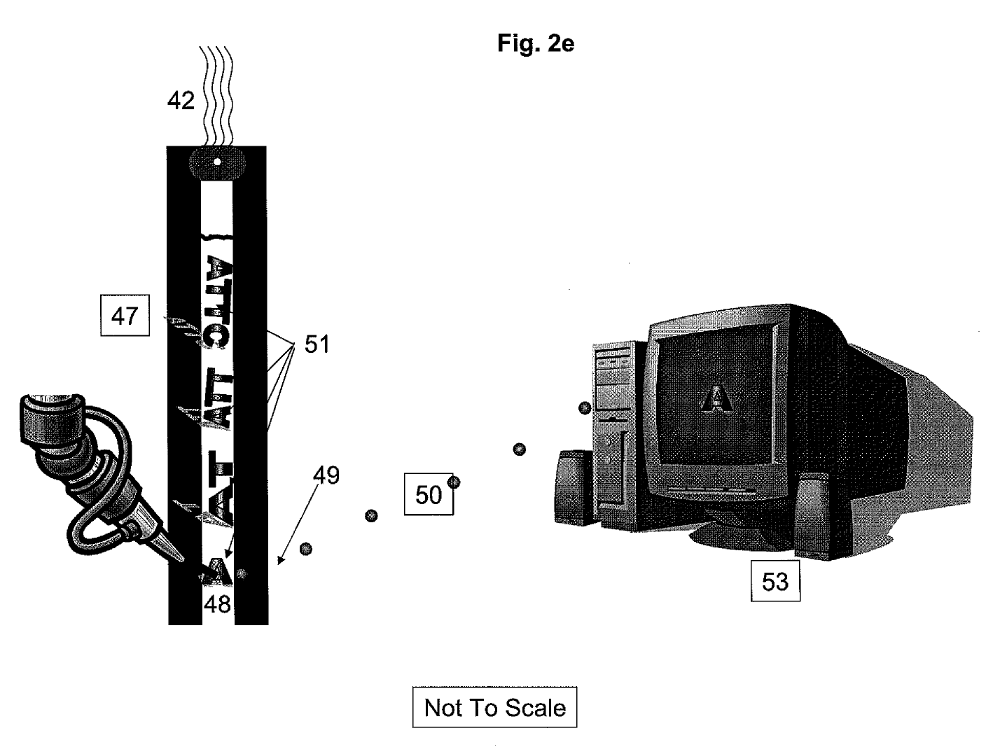

The method and apparatus disclosed herein support the previously defined goals. The method of this invention is illustrated in FIG. 1a and FIG. 2a. The steps of the method include random digestion of genomic DNA into segments using, for example, restriction enzymes. Established genome sequences may be used to produce enzyme restriction maps and to guide restriction enzyme selection process. After the digestion, adaptor ligation is performed of DNA fragments with pre-defined sequences to the segments produced in the digestion. The pre-defined sequences are selected to contain stretches of DNA that can be primed with a single set of DNA primers. The next step is amplification by Polymerase Chain Reaction (PCR) of the segments generated after adaptor ligation (for example 200-1200 bp (base pair) segments of double strand DNA,) The PCR amplification of all segments is performed using a single set of DNA primers. The mixture of amplified segments is then dispensed into a plurality of locations (for example ˜10,000,000 locations), as illustrated in FIG. 2b, forming an array 40 for further specific processing at each individual location 41. The array can be subdivided into smaller groups of equal or different dimensions, spaced evenly or randomly. An array 40 can contain few, a small number (thousands), or millions of locations 41.

At each location 41, as shown in detail 41a, specific oligonucleotides (single strand DNA) 42 are immobilized in advance by methods well known to those skilled in the art. One such method is photolithography, which is described in detail later in this section. The unique sequences of the immobilized DNA at each location are designed to enable hybridization with unique DNA (appear only once in the sequenced genome) of one of the segments from among the digested genomic DNA segments as to capture at each location only specific such segments.

A further illustration of the concepts taught herein is shown in FIG. 2c where an array of locations 41 is represented with corresponding channels 47. At each location a different oligonucleotide 42a, 42b, 42c . . . , or a suitable oligonucleotide combination is present. The DNA sequences in the figures are shown for illustrative purposes only and are non-limiting examples. The channels 47 designate any combination of structural elements at each location 41 that may provide the necessary functionality further disclosed herein. Although the figure shows “standing” “pipes”, other geometries can be used as well, some of which are presented as embodiments disclosed herein. The channels may contain means designated as, and having the functionality of, gates 46, reservoirs 45, and electrophoresis gel materials 48, FIG. 2b.

In the next step the amplified DNA segments are hybridized to the immobilized DNA fragments at each location, first by denaturating the double strand DNA(dsDNA) of the segments into separate single strands (ssDNA) at a high temperature, followed by annealing the separated strands with the immobilized DNA at a lower temperature, or a sequence of temperature steps, thus causing the hybridization of specific ssDNA to be selected from the DNA mix with its complementary immobilized DNA at each location. This process may be accomplished in a single step in all locations in parallel, or through a sequence of steps in a predefined succession in each or in a subset of locations. Each location is uniquely defined and chemically isolated from other locations, FIG. 1b, by different methods, including those disclosed as preferred embodiments herein, and the interactions between the locations are prevented by one of many possible methods, some of which are subsequently described as preferred embodiments.

The next step includes dislodging the captured segments, for example by high temperature denaturation, from the immobilized oligonucleotides, each at its specific location. The step may be preceded by a “cleaning” or “washing” step for removing unattached segments from each location and its surroundings. This process can be done in few, a small number (thousands), or millions of locations in parallel.

In the following step sequencing reaction components are introduced into all of the locations, in parallel, one by one or in any predefined sequence. As a non-limiting example, the sequencing reaction may include the following ingredients in an appropriate buffer: dislodged captured DNA segments (“Template” DNA,) dNTPs (dATP, dCTP, dUTP and dGTP), primer (an anchor sequence for PCR assay) DNA polymerase, MgCl₂. The forward primer sequence is the same one that was used for the previous amplification stage. In this stage it will serve for the initiation of the sequencing procedure. Some dNTPs present in the solution have their bases chemically modified with fluorescent dyes. Each of the four kinds of bases is modified with a different colored dye, four colors in total. Then, while the locations are still chemically isolated, the necessary environmental conditions are provided as to proceed with the sequencing PCR reactions and with the sequencing, FIG. 1c and FIG. 2d. Location isolation can be achieved by placing all the components into a reservoir 45 region of channel 47 controlled by gate means 46. The sequence of steps described until this point can be repeated as many times as necessary as to accumulate sufficient amounts of substances at each location as might be necessary in some instances. This process can be done in few, a small number (thousands), or millions of locations in parallel.

During a sequencing PCR reaction a chemically modified base is incorporated into the growing strand, the DNA replication process terminates yielding a DNA fragment labeled with a fluorescent dye of one of the four colors. A completed sequencing reaction contains a mixture of colored DNA fragments. The shortest are the length of the primer plus one colored base and the longest fragments are usually between 200 and 1200 bases long, but may become longer as technology evolves.

The DNA molecules produced in the sequencing reaction are subsequently separated by DNA electrophoresis. The DNA is allowed to migrate from said locations within channels associated with these locations. DNA molecules are negatively charged. To promote DNA migration an electric field is applied to each specific channel. The electrophoresis separation takes place in region 48 of channel 47, FIG. 2d. Electrophoretic separation occurs in parallel in some or all channels at a time in any sequence as appropriate or predefined. The DNA fragments are separated based on size as they migrate. The channels may be filled with a gel to facilitate the size-based separation. Shorter fragments of DNA move more rapidly through the gel than larger fragments do. As sufficient degree of fragment separation is achieved by the time DNA arrives at a certain segment 49, FIG. 2e, of channel 47, fluorescence 50 of the advancing fragments 51 is induced. This fluorescence can be induced in all channels in parallel, or in one channel at a time in any predefined sequence. The dye colors of each separated fluorescently tagged DNA fragment are then established using their characteristic light emission profile. The fluorescence thus generated can be detected by any suitable sensor type known by those skilled in the art, by applying one or many such sensors at all suitable channel segments in parallel, or one at a time in any preferred sequence.

The fluorescence information is received and analyzed by processing unit 53 to provide the DNA genome sequence. The sequences of DNA segments captured at each location are determined by sequentially reading the colors of fluorescently labeled DNA as it migrates through the corresponding channels. A computer program receives the sequencing information from all the channels and integrates the information collected from individual sequencing. It detects the sequence areas where segments overlap, to align the sequence segments back together. Many overlapping sequencing reads are needed to reveal the contiguous sequence of the original stretch of genomic DNA. On average, every base pair of human DNA will be sequenced multiple times, depending on the length of the segment. Some segments of DNA are easier to read and need to be sequenced less often to obtain a high-fidelity sequence. Other stretches need to be analyzed more exhaustively to obtain a finished high-fidelity sequence.

To facilitate the implementation of the method of this invention an apparatus is disclosed herein. The apparatus is equipped with an array of isolated locations that receive genomic DNA solution mixture after digestion, adaptor ligation and PCR amplification. These locations contain immobilized DNA strands for capturing specific DNA segments from the DNA solution mixture. After specific DNA selection is completed, DNA sequencing components are added to the locations and sequencing reactions are allowed to proceed. An array of channels is associated with the locations for performing DNA separation after sequencing reactions are completed. To enable DNA separation in the channels by electrophoresis the apparatus is equipped with electrodes that are placed as to promote DNA migrations along the channels from their entry portions towards their exit portions. Gates are placed at the entry portions of the channels, the gates control the access of DNA from the locations into the channels. The sequences of DNA segments in the channels are established using an optical-electronic system that is placed as to induce and register DNA fluorescence emission, once DNA achieves a sufficient degree of separation in the channels, and a computer system which is connected to the optical-electronic system. The computer system controls the operation of the optical-electronic system, collects the fluorescence emission information from it, and processes the information to produce DNA sequence data.

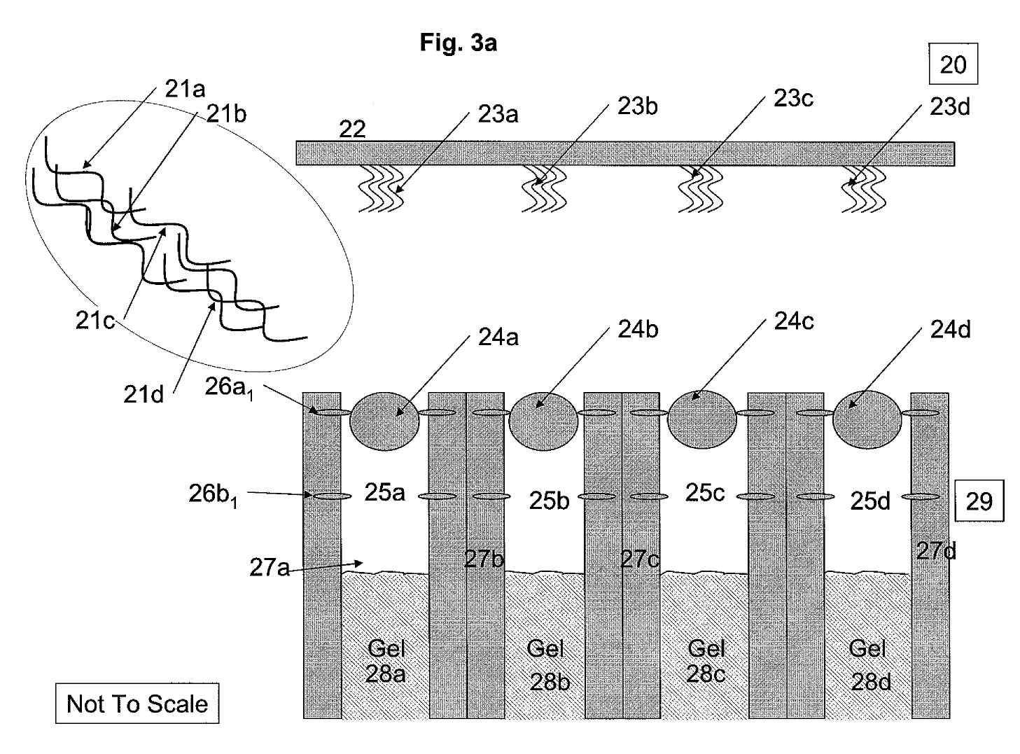

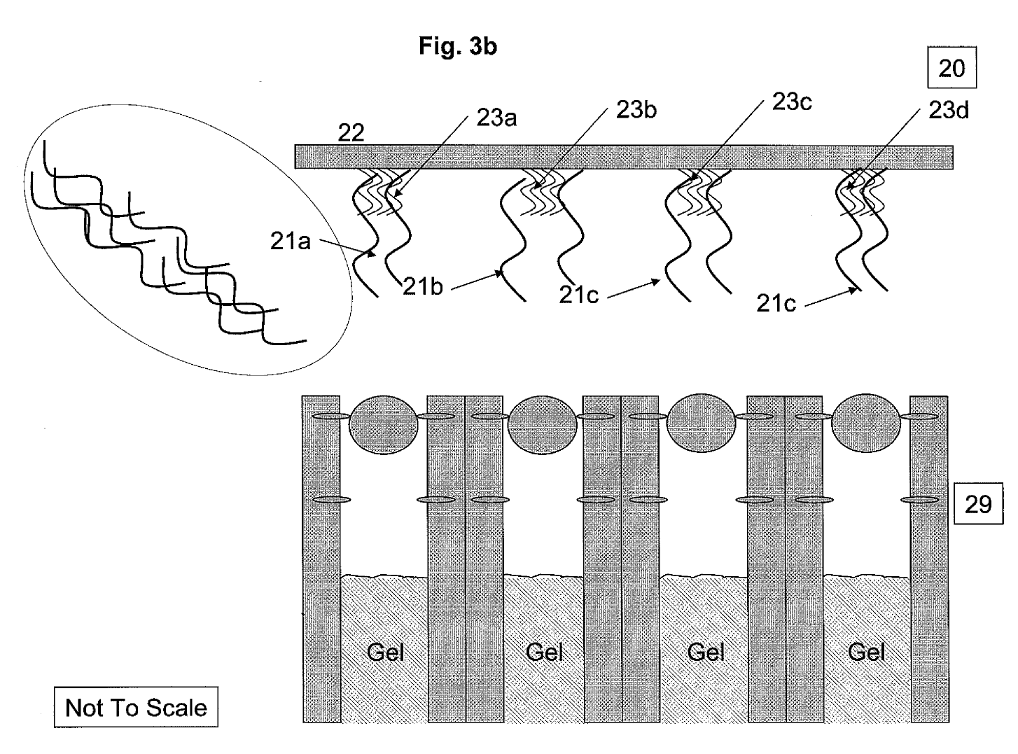

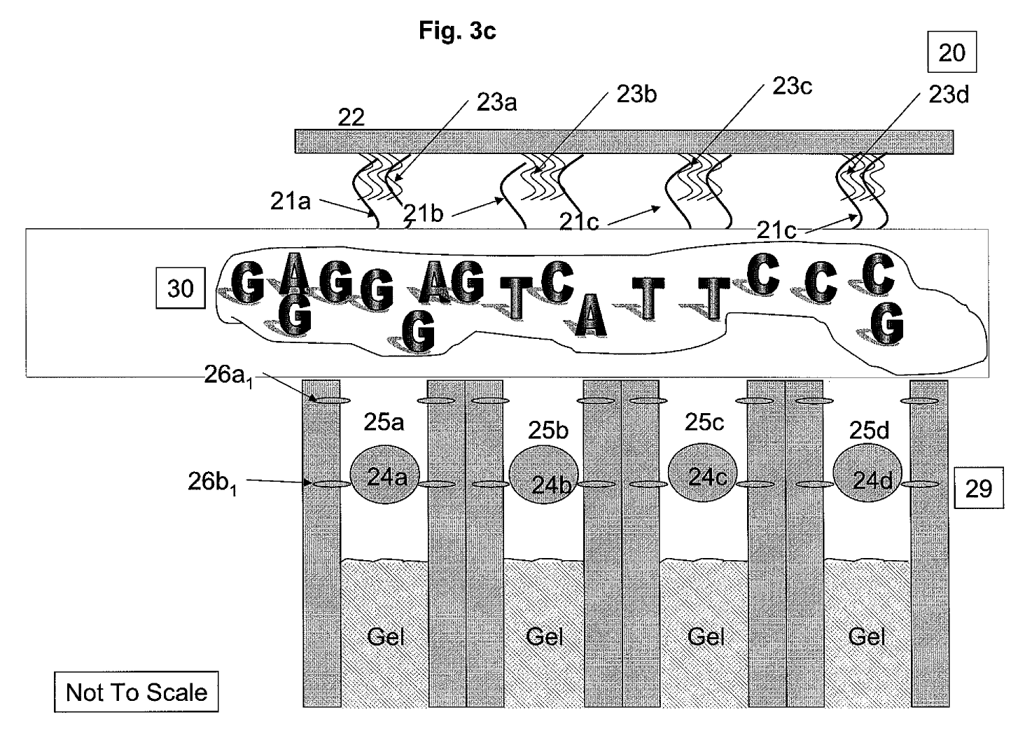

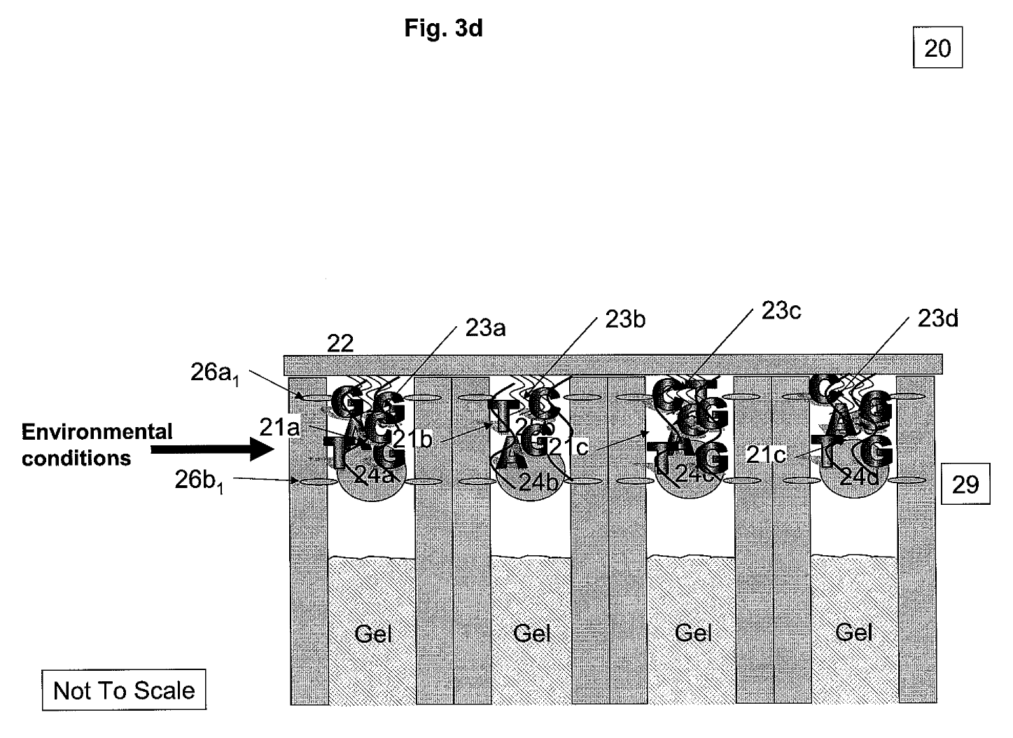

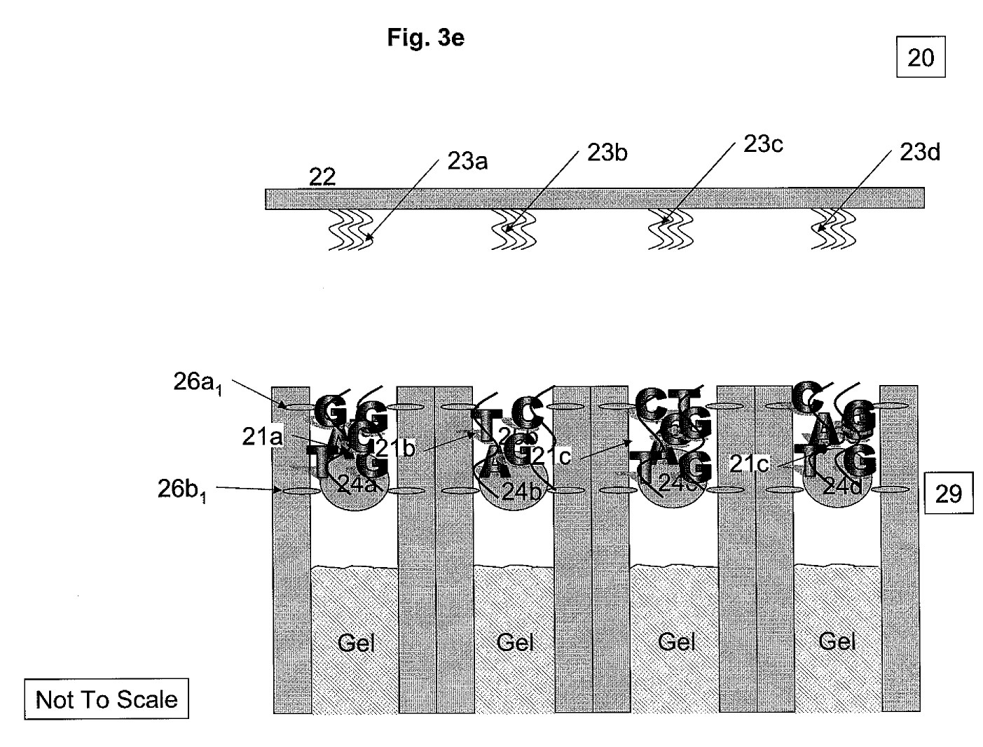

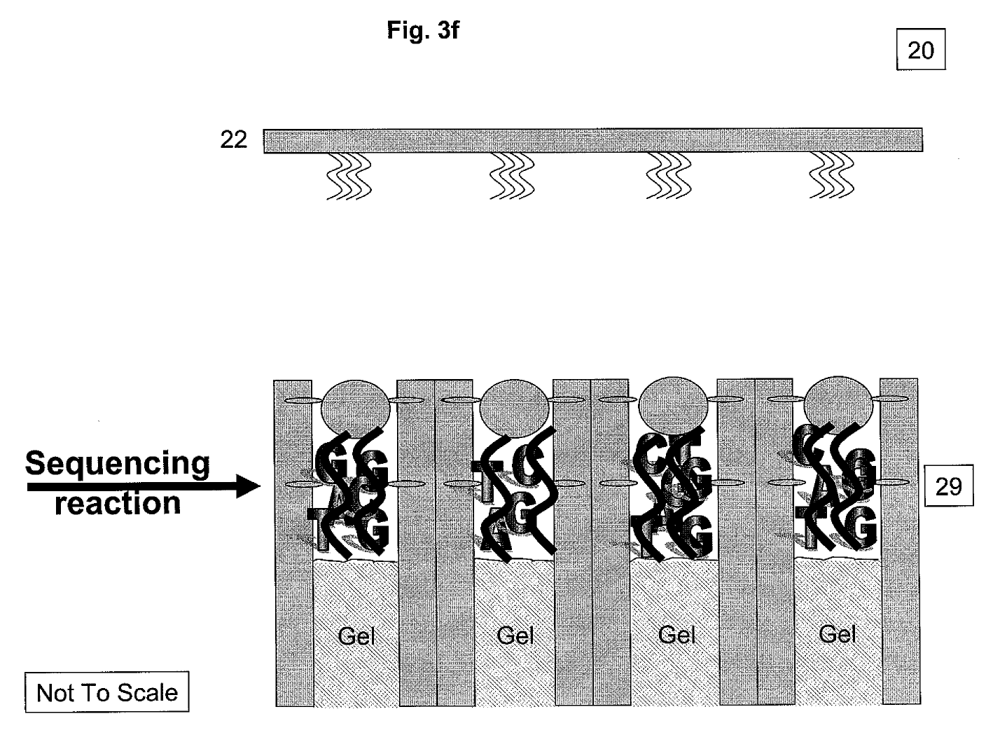

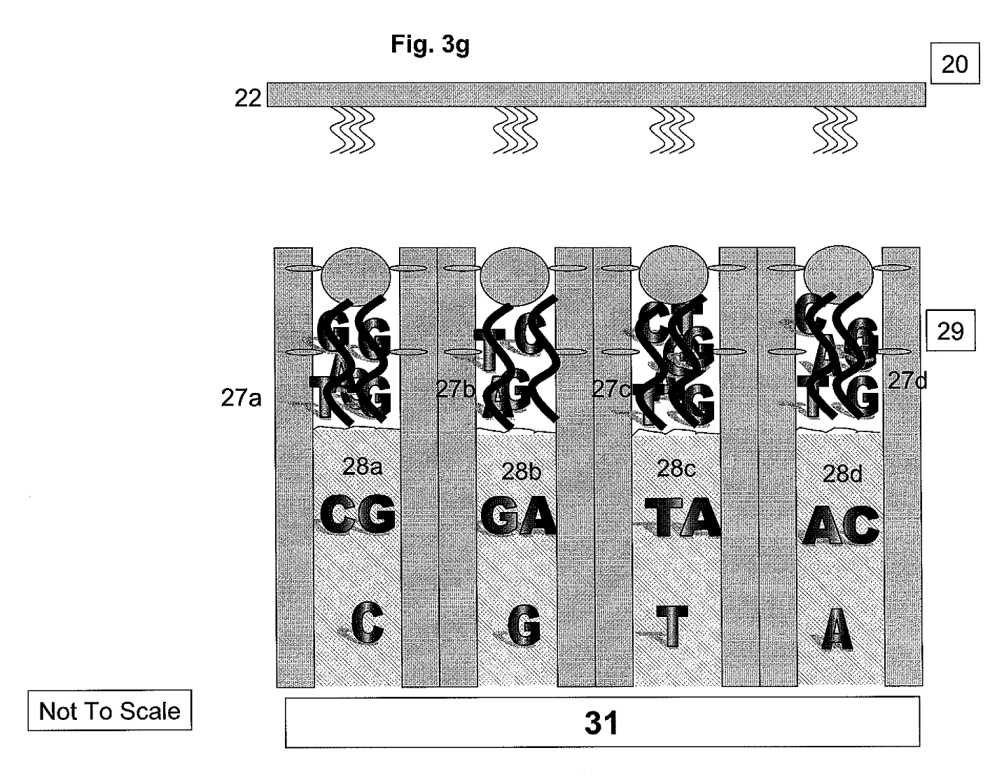

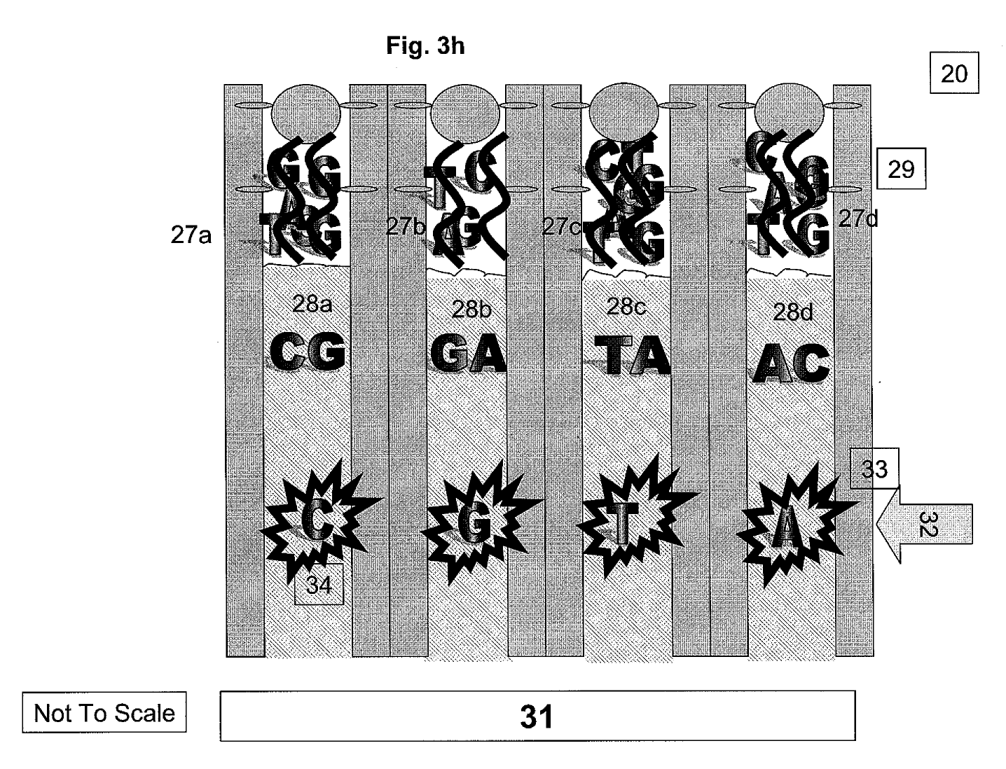

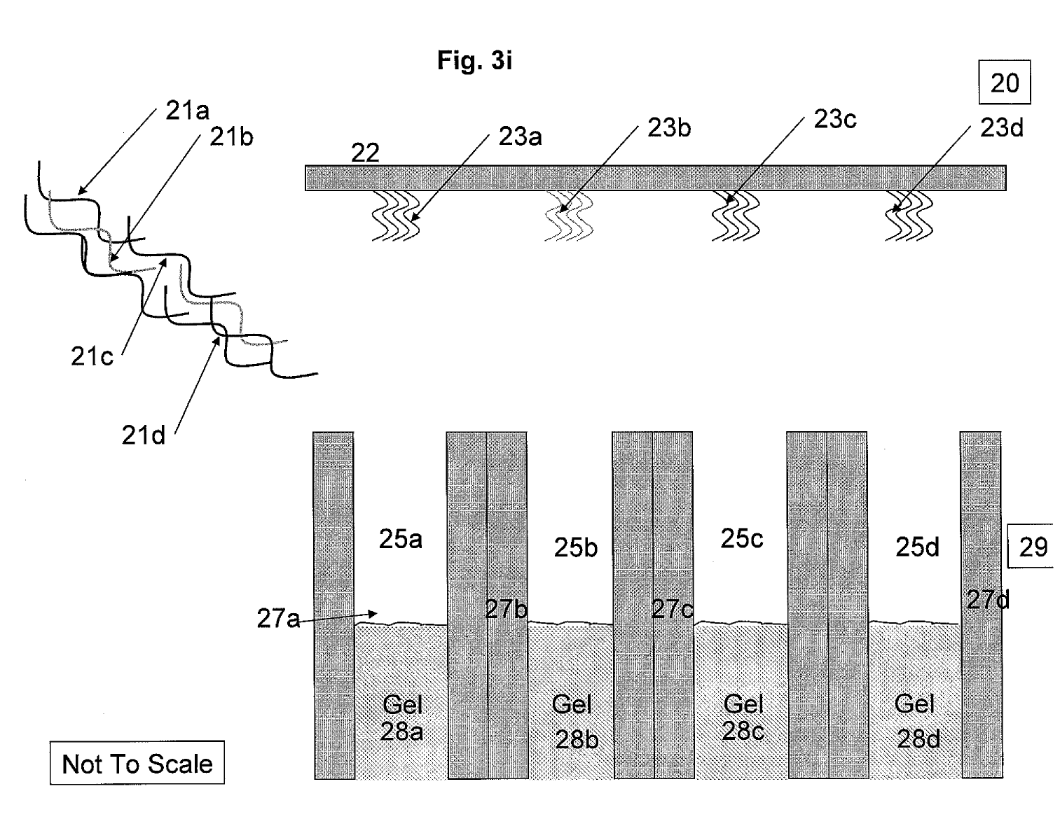

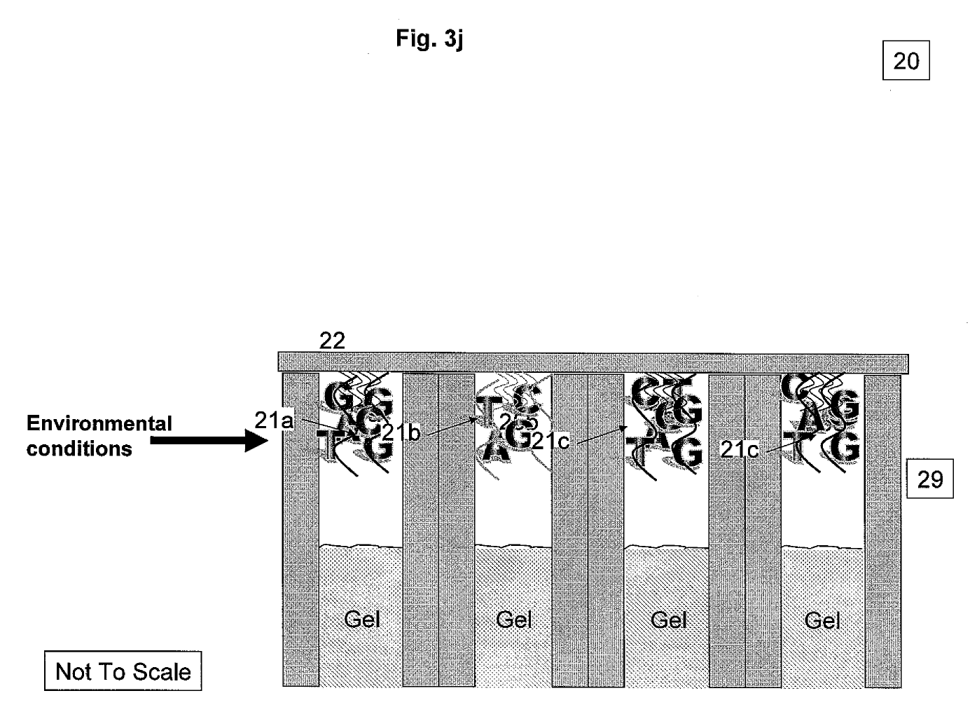

One embodiment of the apparatus is illustrated in FIG. 3a, FIG. 3b, FIG. 3c, FIG. 3d, FIG. 3e, FIG. 3f, FIG. 3g, FIG. 3h, FIG. 3i and FIG. 3j. In the embodiment the apparatus comprises a holding structure 22 that holds oligonucleotides 23 attached at specific positions, FIG. 3a. The placement of the specific oligonucleotides at the specific positions, 23a, 23b, . . . , can be achieved by methods disclosed in the examples below or other equivalent methods known in the art. The embodiment further comprises an array of reservoirs 25 at the locations for aliquoting DNA. In the reservoirs none, one or more gate openings are provided. Gate openings 26a (1,2,3,4, . . . ), 26b (1,2,3,4, . . . ), provide access from different compartments to the reservoir 25. Specifically, in the current embodiment gate openings 26a control access to the oligonucleotide holding zones, and 26b control access to channel sides 27. Channels 27 are filled with gel 28. The gate openings 26 can be opened or closed each one irrespective of the other, or in combination with each other. In the current embodiment, when one gate is opened the other one is closed and vice versa. In general, this and other gating action can be achieved through a variety of gate types, some embodiments for which are further disclosed herein. In the current embodiment, the gating action is achieved with a “ball” component 24. When gate 24 is at gate opening 26a, it closes the accesses from one side of the reservoir and keeps the other side corresponding to gate opening 26b opened and vice versa. When gate 24 closes opening 26a, amplified DNA segments 21 can fill the gap between holding structure 22 and array 29 comprising the channels 27 with the reservoirs 25 and the gates 24, as previously described. While exposing segments 21 to oligonucleotides 23 during a high temperature denaturation and a lower temperature annealing as shown in FIG. 3b, each segment (in this case ssDNA) 21a, 21b, etc. hybridizes with a complementary oligonucleotide 23a, 23b, etc. In this way segment or segment combination 21a binds to oligonucleotide or oligonucleotide combination 23a and so forth.

After removing the segments solution 21, FIG. 3c, and an optional cleaning stage, the sequencing reaction components 30 (as detailed earlier) are introduced into the apparatus 20 between the plate 22 and array 29. The gates 24 that were previously closing opening 26a are now actuated as to close opening 26b, thus exposing reservoir 25 through opening 26a to the sequencing reaction components 30. At this stage plate 22 is actuated as to close reservoir 25 filled with sequencing reaction components 30 and exposed to the oligonucleotides 23 and its complimentary attached segments 21, FIG. 3d. In this way reservoir 25a will contain some amount of the sequencing reaction components 30, as well as the corresponding bound oligonucleotides 23a and segments 21a, likewise for reservoirs 25b with oligonucleotides 23b and segments 21b, etc. Applying environmental conditions, such as high temperature, segments 21 are dislodged from the complementary oligonucleotides 23 into specific respective reservoirs 25. Subsequently, plate 22, now holding only oligonucleotides 23, is retracted from reservoir 25. Segments 21 mixed with sequencing reaction components 30 are left in their respective reservoirs 25, as shown in FIG. 3e. Gates 24 are then actuated again as to close openings 26 and prevent the accumulated materials from escaping reservoirs 25, FIG. 3f. The sequence described until this point can be repeated as many times as necessary as to accumulate required amounts of substances at each location.

Once the above sequence is completed, FIG. 3f, sequencing reactions are allowed to proceed. After sequencing reactions are completed, FIG. 3g, an electric field 31 is applied between the termini of channels 27 as to induce electrophoresis motion of the species intended for sequencing. The field induces separation according to species size of the fluorescently tagged fragments through the lengths of channels 27, the separation may be better facilitated if the channels are filled with gel 28. The electric field in this embodiment is induced by placing electrodes at appropriated positions know to those skilled in the art. These positions can be on the inner or outer faces of channels 27. The electrodes can be placed at the gate openings or at the channel ends or at any other location or geometry as may be applicable to provide the required electrophoresis. During the electrophoresis separation some segments of channels 27, as shown in FIG. 3h, are illuminated with light beam 32. The light beam induces fluorescence 34 of the dyes attached to the sequenced species as they arrive at segments 33. The emitted fluorescence is registered by a detection device know to those skilled in the art, for example a CCD device, CMOS device, etc. Characteristics of the spectra of emitted fluorescence, including wavelengths and durations of fluorescence emission, and the sequence in which such characteristics appear are analyzed to produce genomic DNA sequences.

As previously indicated, the flow of substances in and out of the reservoirs can be controlled by a gate structure, some representative embodiments of which are disclosed herein, or without utilizing such gate structures. In an apparatus embodiment without a gate structure access to all the reservoirs can be controlled in parallel by a cover plate that opens or closes all the locations at the same time. Such an embodiment is represented in FIG. 3i and FIG. 3j, reservoir access control is performed only by plate 22. In FIG. 3i the plate is shown in its open position when the substances are not in the reservoirs (similar to FIG. 3a but without gates 24). In FIG. 3j the plate is shown in its closed position while the substances are inside the reservoirs and isolated from each other by plate 22 (similar to FIG. 3d without gates 24). It is obvious to those skilled in the art that an inverse embodiment can also be used, where plate 22 is not used and only gate or gates 24 or equivalent gate structures are utilized. For such a case oligonucleotides 23 can be placed in positions as further disclosure herein.

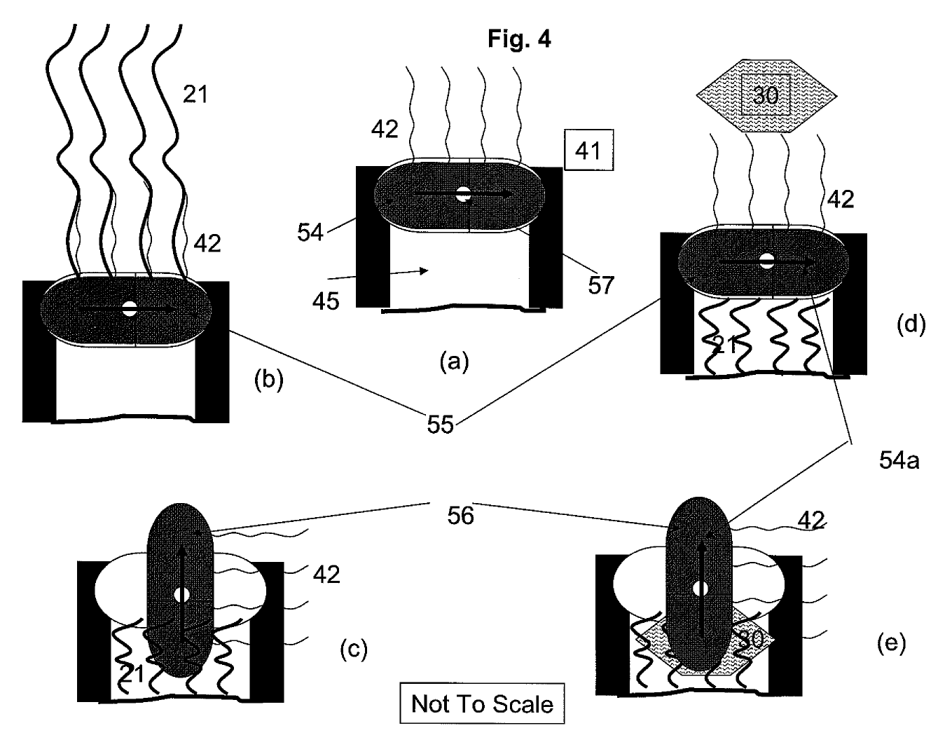

Further embodiments of gates 46 are taught herein to facilitate the control of the substances at each location 41, FIG. 2b. One embodiment of such gates consists of a small rotating gate 54 that swivels between positions 55 and 56 of location 41, FIG. 4. In a preferred embodiment gate 54 can rotate about a hinge or about an elastic element that permits the rotation around an axis 57 to an extent necessary for the application. Gate 54 can be attached from its axis of symmetry or any other axis. The gate may have a directional preference of some of its qualities 54a, for example magnetic or electric dipoles. The gate can open and close the access to reservoir 45 at location 41, if desirable it can in the same action supply the substances explained previously to a nearby location. In the preferred embodiment gate 54 rotates around axis 57, FIG. 4. In this embodiment, oligonucleotides 42 are attached to the “outer” side of the gate 54 (“outer” is with respect to reservoir 45 and is used for clarity of explanation only). When oligonucleotides 42 are exposed to amplified DNA segments 21, FIG. 4b, complementary segments can hybridize to oligonucleotides 42 immobilized on gate 54. After the segments are hybridized to oligonucleotides 42, gate 54 is rotated to a second position 56, FIG. 4c. At position 56 environmental conditions are induced as to cause segments 21 associated with oligonucleotides 42 to dislodge into reservoir 45. In the next step, gate 54 is rotated back to position 55, closing reservoir 45, thus isolating segments 21 from the outside environment. This sequence can be repeated several times as to accumulate a required amount of segments 21 in reservoir 45. Then sequencing reaction components 30 are provided to location 41, FIG. 4d, and gate 54 is rotated to position 56, FIG. 4e, allowing the sequencing reaction components to enter reservoir 45. At the next step gate 54 is rotated to position 55, thus closing reservoir 45 and isolating it from the outside environment. At this stage the sequencing reaction and subsequent steps described earlier can proceed. In the embodiment illustrated in FIG. 4, the selection step that can be implemented by rotating the gate by an angle, for example 90 degrees, as to put the oligonucleotides 23 and the hybridized segments 21 into the controlled area of the reservoir The segments can then be dislodged from the oligonucleotides, for example using high temperature, thus entering the controlled areas of the reservoir. Since only one segment type or a predefined combination of segment types is attached at each location only a specific segment or segment combination is stored at each location reservoir. The next step can be rotating back the gate to its closed position and expose the oligonucleotides to the “outer” environment, readying it to collect another amount of specific segments as to enlarge the amount of segments in the location reservoir or to isolate the reservoir in preparation to the next steps as required.

Gate actuation, in general, can be achieved by many possible methods, which are based on electrostatic, electromagnetic, thermal, piezoelectricity, or equivalent principals. One such method is electrostatic actuation. If one of the materials composing the gate is conductive and connected to a source of electricity, for example a battery, and another electrode generating an adequate electrostatic field is placed in the vicinity, the resulting electrostatic force can be used to open or close the gate (closing or opening refers to any motion of the gate, such as elevating it, rotating it, flexing it, and so forth.)

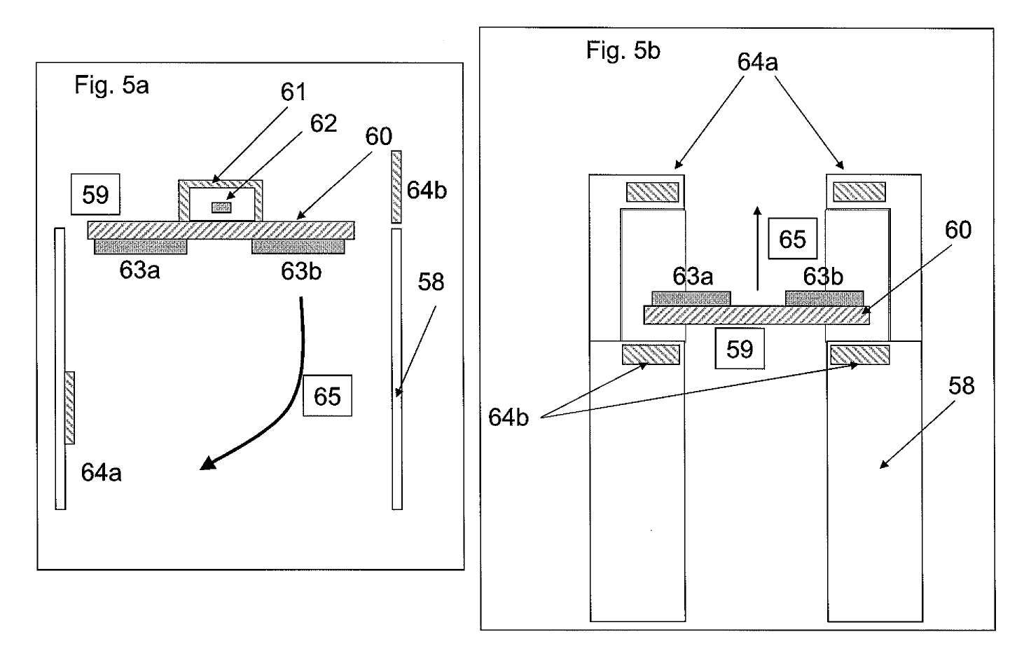

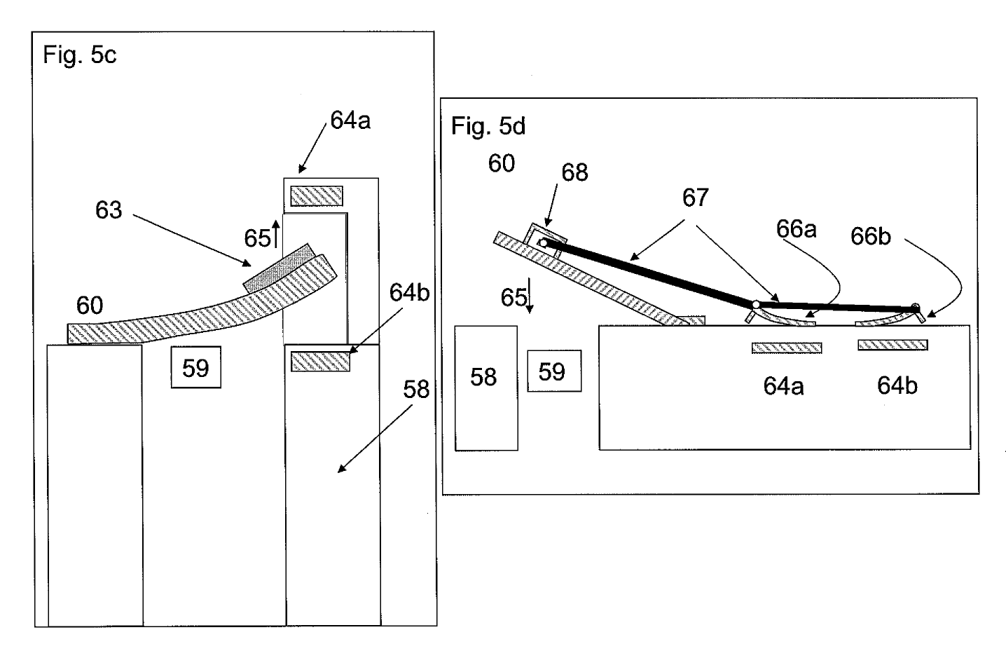

One embodiment of the actuated gate configuration is represented in a cross section in FIG. 5a, where channel 58 contains gate 59 composed of a holding plate 60 and attached by a hinge 61 around axis 62, which is held at a fixed position. Gate plate 60 holds one or more electrodes 63, electrodes 64 are positioned at the channel, either internally or externally. When applying opposite potential between the sets of electrodes, gate 59 is forced to rotate, thus opening or closing access to the channel. For example, if opposite charges are induced between electrode 64a and 63b and/or electrodes 63a and 64b, the gate will rotate in direction 65. Other electrode placements and configurations that achieve the desired gate function can be implemented based on these teachings by those skilled in the art.

Another embodiment of electrostatic actuation is shown in FIG. 5b. Channel 58 contains gate 59 composed of holding plate 60 on which electrodes 63 are placed, electrodes 64 are positioned at the channel, either internally or externally. When applying opposite potential between the sets of electrodes, gate 59 is forced to elevate (upward in the figure) or retract (downward in the figure,) thus opening or closing the gate. For example, if opposite charges are induced between electrodes 63 and 64a, the gate will move in direction 65. In another actuation scheme, if opposite charges are induced between electrodes 63 and 64b, the gate will move in the opposite of direction 65.

Another embodiment of electrostatic gate actuation is shown in FIG. 5c, where channel 58 contains gate 59 composed of holding plate 60 on which electrodes 63 are placed. Plate 60 can be pre-shaped or pre-stressed to obtain a curved shape, as in FIG. 5c, or can be flat originally and attain a curved shape due to the actuation action. Electrodes 64 are positioned at the channel, either internally or externally. When applying an opposite potential between the sets of electrodes, gate 59 is forced to flex up (in the figure) or flatten down (in the figure,) thus opening or closing the access to the channel. For example, if opposite charges are induced between electrodes 63 and 64a, the gate will bend in direction 65. In another actuation scheme, if opposite charges are induced between electrodes 63 and 64b the gate will bend/flatten in the opposite of direction 65.

Another embodiment of electrostatic gate actuation is shown in FIG. 5d, where access to channel 58 is controlled by gate 59 comprised of plate 60. Plate 60 can be rotating, flexing, or of another similarly functioning type. In the example shown on FIG. 5d, the plate is shown to flex. Plate 60 is attached through hinge (or spring) 68 and links 67 to scratch drive actuators 66. SCRATCH drive actuators are activated using electrodes 64. While energizing electrode 64a, scratch actuator 66a will actuate and move plate 60 through link 67 and hinge 68 in direction 65. If electrode 64b is energized, then scratch drive actuator 66b is actuated as to move plate 60 in the direction opposite to 65. In this explanation, scratch drive actuators 66 are represented symbolically with an understanding that they are actually more complex and may contain more components than shown in the figure. Those skilled in the art can substitute scratch drive actuators with other actuators, such as comb drive actuators etc., to achieve the same or similar actuating function. It can also be understood that other embodiments of the gates and actuators can utilize any actuator type, one or several of them, to actuate one or many gates together by linking them with appropriated links and levers.

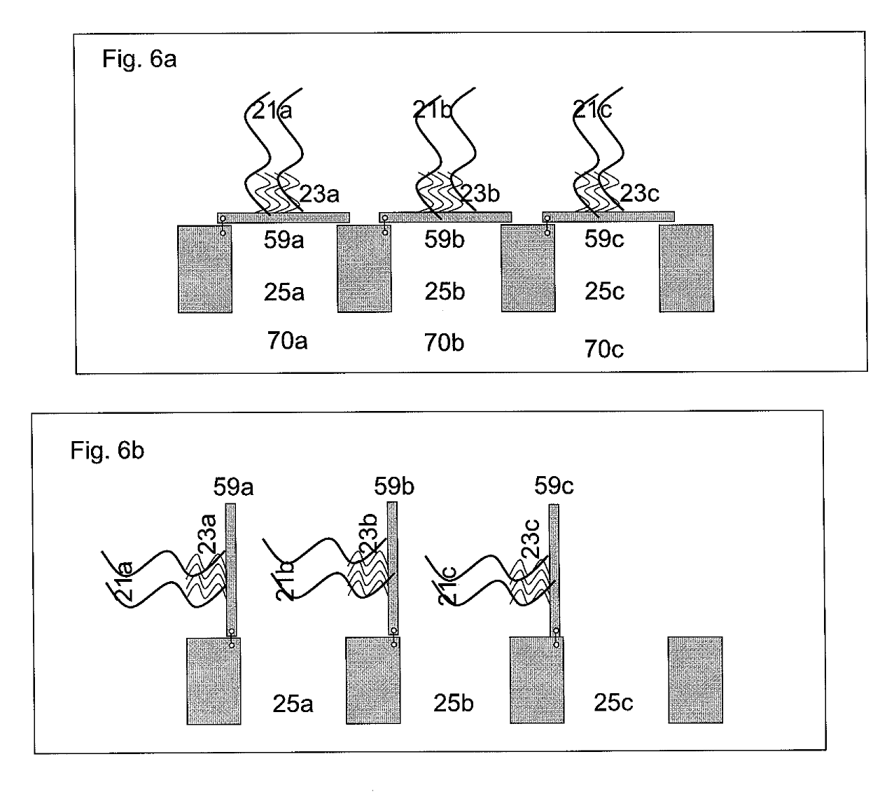

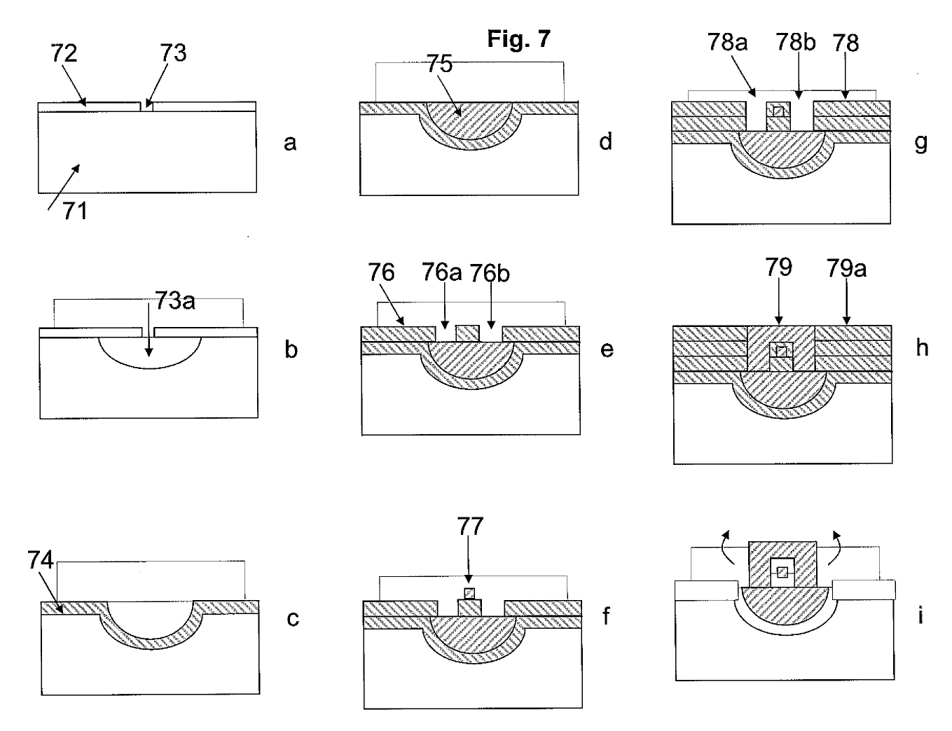

Another not limiting embodiment of the gate action can be a gate with a hinge not centered in its symmetry axis, FIG. 6. In such case, when the gate opens, the oligonucleotides and their respective captured segments, as one of many options, can be made to emerge on the zone controlled by a nearby reservoir, not the one that the gate is closing. The other steps are as previously explained, but each gate holds the fragments and oligonucleotides belonging to a neighboring or even further away location. For example, this method can be useful where swiveling gates might not be appropriate or when space is at premium. To exemplify such an embodiment, FIG. 6 shows locations 70 on which gates 59 are place. Gates 59 contain immobilized oligonucleotides 23 with hybridized complimentary segments 21. Gates 59 transit into their open position as to allow access to reservoirs 25 to receive segments 21, FIG. 6b. In this embodiment gate 59a opens reservoir 25a of location 70a, but receives segments 21b hybridized with oligonucleotides 23b immobilized on gate 59b, which opens reservoir 25b of location 70b. At the same time, location 70b will receive into reservoir 25b segments 21c hybridized with oligonucleotides 23c, which are immobilized on gate 59c, and so forth. Following previous teachings, actuation of these gates can be achieved electrostatically. The electrostatic actuation can be applied by connecting the gates through hinges or flexible elements, fixtures to electrostatic actuators placed at the gate locations, nearby locations, or even distant to the gate location. Actuators can be associated with a specific gate, or can be moved between gates requiring it. Many gates can use one actuator, or a single gate can use one or many actuators at the same time, in any configuration or timing possible as to achieve the required motion. The electrostatic actuators mentioned can be, but are not limited to, scratch drive actuators, comb drive actuators, or another suitable actuator.

The fabrication of the gates, reservoirs and actuators can be achieved utilizing many exiting methods. Due to dimensions and techniques, these existing methods can cumulatively be describes as MEMS technology and its extensive toolset. For example, a gate of one of the embodiments can be fabricated utilizing the sequence shown in FIG. 7. The figure is not to scale and is only an illustrative cross section (side view) of the fabrication steps, The fabrication begins with selecting a substrate 71. The substrate can be a Si wafer, or a quartz wafer, or even a bundle of optical fibers, or other fibers prefabricated from the required materials. In the interest of explanation simplicity we will refer to the substrate as made of a Si wafer. A hard mask 72, for example of LPCVD SiN, is deposited on top of the substrate, and an opening 73 is made by standard photolithography and etching methods. The Si is isotropically (wet or dry) etched through mask 73 as to form a hemispherical cavity 73a, FIG. 7b. The removal of the hard mask 72 is followed by the deposition of what will become a sacrificial layer 74. This layer can be made of many materials, for example it can be made of Si oxide. The next step is the filling of cavity 73a with some structural material and polishing it, as to flatten its surface to form the gate plate 75. The plate can be made of various materials as to provide desired qualities. Plate 75 can be made of metal, such as Au, Al, Cu, Ni, or polymers, Poly-Si, Nitride, etc. It also can be made of a combination of materials, or several steps can be added after its fabrication as to form additional structures on top of it. It can be a part of the actuator, or the actuator can be fabricated on top of it. In the current example, plate 75 can be made of gold. In the next step another sacrificial layer is deposited and patterned, the layer can be made of the same material as layer 74 or of a different material. For instance, sacrificial layer 76 can be made of a photopolymer and patterned to have openings 76a and 76b that will serve as molds of a hinge. Openings 76a and 76b can be made at this stage or at the stage shown in FIG. 7g together with layer 78 and openings 78a and 78b. In FIG. 7f, hinge axis 77 is fabricated by, for example, gold electroplating. This hinge is made to be fixed at a static place. The static place can be on the original substrate, or on another structure of the completed device on which the axis is later attached, or attached to other parts of the device at a later fabrication step, not described here but understood by those skilled in the art. Another layer of sacrificial material 78 is placed and openings 78a and 78b are made, openings 76a and 76b can be made at this stage as well. Finally, hinge loop 79 is made by, for example, gold electroplating through sacrificial layer mold 79a and openings 76a, 76b, 78a and 78b. The last step is dissolving sacrificial layers 79a, 78, 76 and 74 as to release the gate plate and leave it hanging on axis 77 and loop 79, free to rotate in the directions shown by the arrows, FIG. 7i.

Similar methods can be applied to fabricate the other embodiments of gates and actuators appropriated to be implemented in the teachings of this disclosure, or described through this disclosure. The plate can be a part of the actuator, or the actuator can be built on it, or combined into it. The gates and the actuators fabricated as explained can be of any type: thermal, electrostatic, and so forth. Notably, if the plate 75 material is made of nickel, or of other magnetic or ferromagnetic material, the actuation can be achieved electromagnetically, or as a combination of actuation methods.

As another preferred embodiment, the gate can be made of a ferromagnetic or magnetic material and thus can be actuated by an internal or external magnetic field. Referring to FIG. 4, gate 54 can be made from a magnetic material that is placed, or is part of the gate fabrication stages, as explained earlier in relation to FIG. 7. In this embodiment magnetic actuation can be utilized. The gate will have a magnetic dipole 54a caused by the magnetic material. Applying a magnetic field to one or many locations at an angle to dipole 54a will actuate gate 54 from position 55 to position 56. Also, applying a magnetic field at an angle (that may have a different orientation) to the dipole 54b will actuate the gate from position 56 to position 55. The creation of the dipole can be achieved by using a fabricated inductor, or using ferromagnetic materials, eddy currents, etc.

While using magnetic fields other realizations of the gates disclosed or referred to in this disclosure can be adapted to work under magnetic actuation. Such embodiments are, but not limited to, the embodiment shown in FIG. 3a, where gate element 24 can be made of a magnetic or a ferromagnetic material, and acquire magnetic properties by any known method, such as an inductor loop, eddy current, etc. Ball 24 in FIG. 3a can thus be switched between openings 26a and 26b by applying an appropriate magnetic field to each location or to all the locations as to provide for the required actuation schema. In configurations presented in FIG. 5a-c and FIG. 6a-b, electrostatic actuation can be directly replaced with magnetic actuation where the electrodes or plates can be partially or entirely replaced with magnetic or ferromagnetic elements, and fields be placed as appropriate.

As disclosed earlier, the actuation can be performed also by other methods, such as by electromagnetic actuation induced force. This can be applied to the configurations described previously for electrostatic actuators, whereby the gate can be made of or contain a magnetic or a ferromagnetic material, or the magnetic field of the gate can be induced by fabricating a conductive “loop” or inductor at the gate and supplying an electric current to it. The magnetic force can be induced using an external magnet, or inductor, or by inducing Lentz (eddy) currents. These forces and configurations can be provided locally at each location, for all the locations at once, or in any predefined combination and timing.

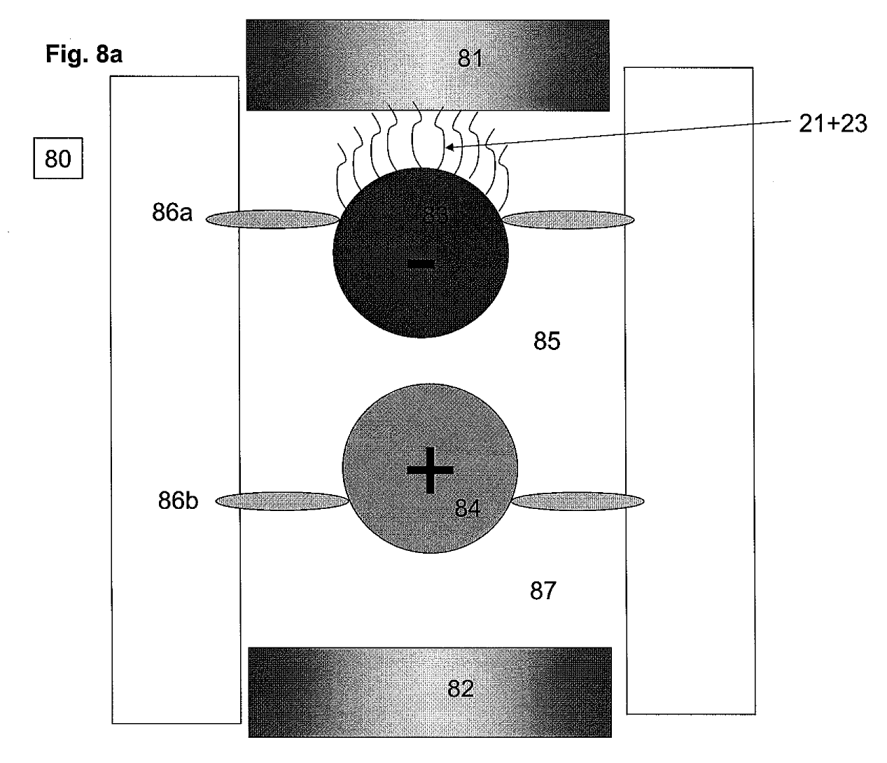

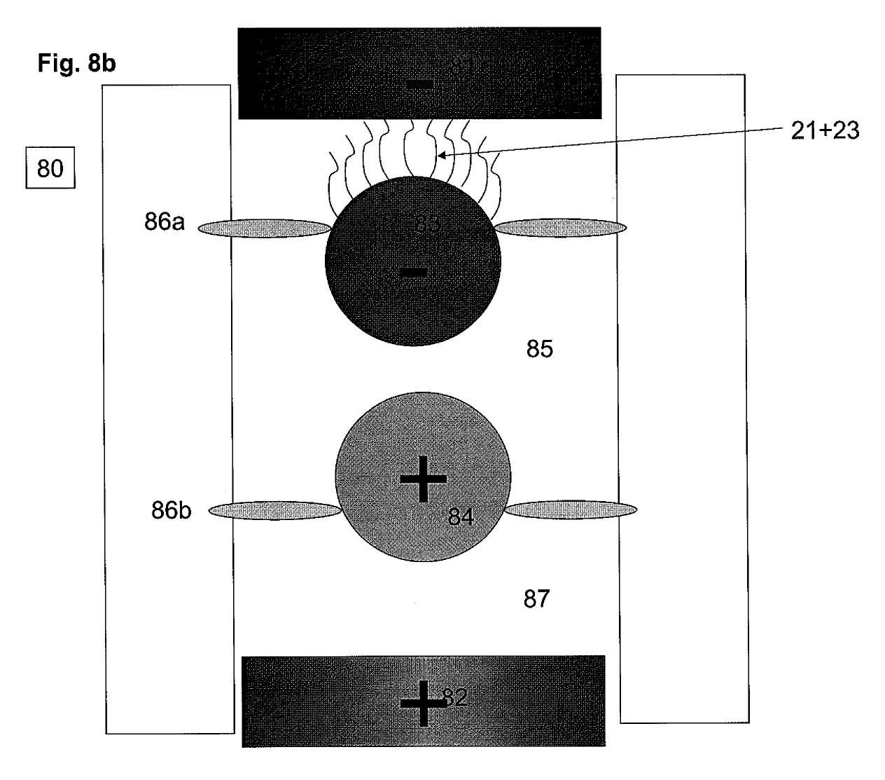

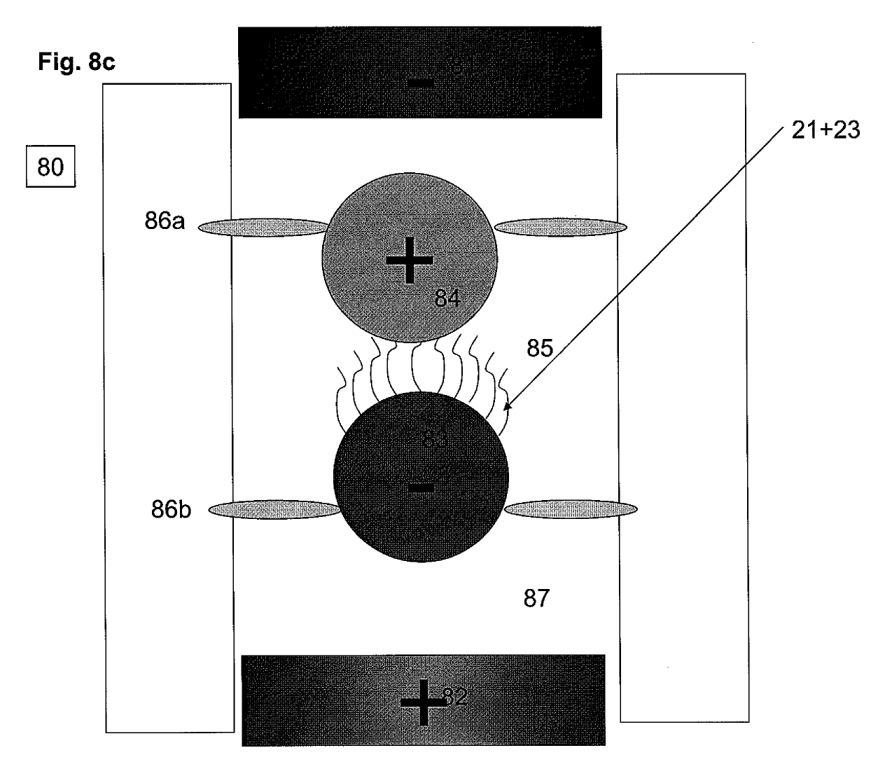

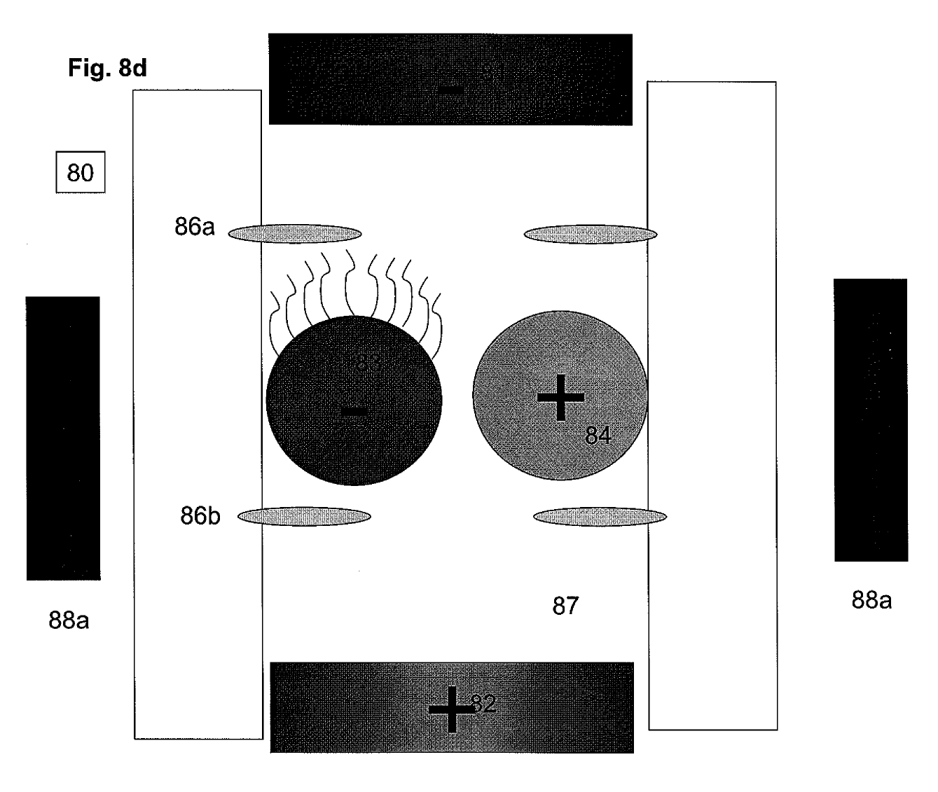

While specifying the actuating methods in all the embodiments throughout this disclosure, it may also be desirable and possible to implement combinations of such actuation methods. Such combinations can be, but are not limited to, an electrostatic and a magnetic combined actuation of a single or many gate elements per location, or at several locations. One embodiment of such a combination is shown in FIG. 8a-d, where gate 80 is shown having two gate elements 83 and 84, closing gate openings 86a and 86b, respectively, isolating reservoir 85 and compartment 87. On gate element 83 oligonucleotides 23 with segments 21 are immobilized. In a preferred embodiment the gate is of a “ball” shape and has electric and/or magnetic properties. In the preferred embodiment of FIG. 8a-d gate element 83 has an electric charge and a specific magnetic dipole referred to as “−” whereas gate element 84 has another charge and a magnetic dipole referred to as “+”. While in the actuation stage of this combined gate embodiment electrodes 81 and 82 are energized with opposite electric, magnetic, or both fields, the magnitude of the electric field in the preferred embodiment should be comparable to the magnitude of the field generated by the charge (or dipole) of the respective closest gate element. In FIG. 8b, electrode 81 will receive a charge referred to as “−”, which is comparable in magnitude to the charge of gate element 83. Electrode 82 will receive a charge referred to as “+”, comparable in magnitude to the charge of gate element 84. This field configuration will cause the gate elements 83 and 84 to be repelled form electrodes 81 and 82, respectively. At the same time, gate elements 83 and 84 will be attracted by electrodes 82 and 81, respectively This will provide the necessary forces to exchange the positions of the gate elements into the state shown in FIG. 8c. At this state the segments 21 can be dislodged from the oligonucleotides 23 as in the other embodiments. In a related preferred embodiment other fields can be applied to actuate the gate elements into other configurations or to improve the function of the gates. For example, a magnetic, electric or both fields can be applied from another position 88a and 88b as in FIG. 8d. This configuration may prevent the association or collision of the gate elements with each other during the transition by separating their possible trajectories. The additional fields can also be used to keep the gate elements, both or part of them, open as to permit introduction of substances from or into reservoir 85 and/or compartment 87. The fields imposed by elements 88 can be applied at the same time, before or after the energizing or de-energizing period of electrodes 81 and 82. Other actuation methods and combinations of methods can be used to actuate the gates, such as thermal actuation, shape memory alloys, bimetals and other thermal actuators, piezoelectric actuators, acoustic as well as other methods known to those skilled in the art.

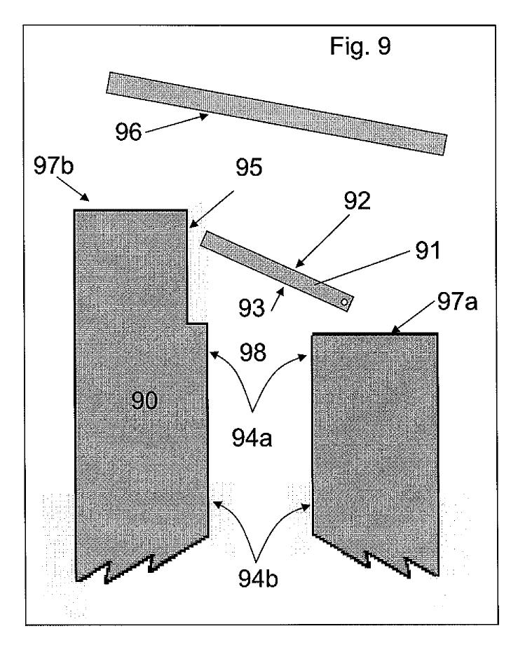

The gate function at each location or at locations nearby is to control the access of predefined specific substances to specific locations, such substances in some cases may differ from each other, while in other cases can be similar or identical to each other. Gate structures or their proximity can also be used for oligonucleotide immobilization. Possible locations for such oligonucleotide immobilization to achieve specific substance selection are illustrated in FIG. 9. The oligonucleotides at location 90 can be attached at its top 92 or at its bottom 93 of gate 91, on the walls 94, 95, bottom 94b or top 94a of the location reservoir 98, in the surrounding areas 96, 97 internal or external to the gate. Oligonucleotides can also be immobilized at the outer side of the gate (42 on top 46 in FIG. 3d, 42 on top 54 in FIG. 4, 21/23 on top 59 in FIG. 6, 21/23 on top 83 in FIG. 8.) When the gate is closed the oligonucleotides are attached to the external side of it (with respect to the gate controlled reservoir) If the oligonucleotides are exposed to the amplified DNA segments, as explained earlier, the segments hybridize with the oligonucleotides.

Following is a procedure for attaching pre-defined oligonucleotides at specific locations applying the photolithography method. Mers are bound at locations 41, see FIG. 2b. Each location 41, or regions thereof, receives none, one or more UV light doses. This light will activate a mer at that location readying it for binding of one nucleotide (A, C, T or G). This procedure may happen in parallel in, for example, 10,000,000 locations. This procedure is repeated for the number of desired nucleotides to construct an oligonucleotide (30-50 nucleotide ssDNA). Each nucleotide contains a base (either: A, C, T, G) and possibly a light sensitive component. A nucleotide is flown over and its sensitized parts will bind the established mer. The next step is exposing locations 41, or regions thereof, to one or more UV light doses. However, the locations exposed at this step may or may not be the same locations or regions as in the previous step. This will prepare the mer or the nucleotides' light sensitive component for linking to the oncoming nucleotide. A nucleotide is flown over the locations, for binding to the exposed locations. This nucleotide may or may not be the same as in the previous or following step. Similar steps follow until oligonucleotides 42 are formed in each location. Since the exposed locations are know during the procedure and the nucleotides are known at each stage, the procedure of oligonucleotide formation at each location is controlled and well known. It should be emphasized that the UV exposure can be done at a specific location, at many locations, and even several times in different locations, as necessary for the fabrication of the attached oligonucleotides. The UV patterning can be done by many methods known by those skilled in the art. One example of such methods is the use of lithography masks or DLP (Digital Light Processor trade mark) arrays to channel the UV light to the intended place.

Still, another embodiment can be realized by a “ball” or other shape (rhomboid, cuboid, pyramid, etc) gate of some, not liming, material such as a metal or magnetic material. When induced by electrostatic or electromagnetic fields from electrodes or inductors near each location or on any close or far location applied onto a single location or many and even all locations together, the ball can move up and down inside the reservoir thus opening and closing it as necessary. In this case the oligonucleotides can be attached to the ball, to the walls of the reservoir, to the top or bottom walls of the reservoir or at many other conceivable locations. In this case when the ball doesn't blocks the reservoir opening, the segments can enter the reservoir and attach to the corresponding oligonucleotides. Once the ball is actuated as to close the reservoir the segments can be detached from the oligonucleotides and left into the control zone of the respective location reservoir. Conversely, another embodiment is implemented while the segments are attached by oligonucleotides at the ball while in the closed state. At this state the reservoir inside is closed but the oligonucleotides are exposed to the outside side where the segments solution exists. At the open state, the ball sinks into the-reservoir. At this moment the segments are released from the ball and left in the inside of the reservoir that is closed by raising the ball.

Still another embodiment for the gate and segment selection method is a surface (22 of FIG. 3a), or a part of a surface, that approaches and separates the reservoir's opening at one or many locations in any sequence, including at the same time. Once the surface approaches, it can contact the outer surface of the reservoir of a location, thus closing it. When the surface detaches and separates it opens the reservoir. In these embodiments the oligonucleotides can be attached to the internal walls of the reservoir, or to said surface. While the surface is separate from the reservoirs, the segments are in contact either with the oligonucleotides at the moving surface or at the walls. When the surface closes the reservoirs and the oligonucleotides and segments are isolated inside the reservoir necessary environment can be created inside the reservoirs to dislodge the segments which then remain in the controlled area of the reservoir. The surface can detach to open the reservoir for further cycles or for additional purposes that might be appropriate. The surface of this embodiment is actuated by any previously detailed method, or any other way easy appreciated by those skilled in the art.

The apparatus that is the subject matter of this invention contains an electrophoresis column section. An electrophoresis channel can be shared by all of the locations or each location can have its own electrophoresis channels. In the embodiment disclosed herein an electrophoresis column is realized at each location. This column can be attached to, or be at the control of the location. Electrophoretic separation can take place in the columns of each of the locations one by one in any sequence, or all in parallel as may be required or applicable. The channels can be unfilled or filled with substances, preferably gels, the same substance for each channel or a different substance in each channel. Where a gel is introduced into a channel, the gel is applied as required for the electrophoresis size separation of the sequenced DNA segments or fluorescently-labeled sequenced DNA fragments. As the sequenced segments or fluorescently-labeled sequenced fragments advance through the channel under the action of the electrophoretic field, smaller DNA fragments advance faster then larger ones. Thus, DNA fragments pass through the last parts or sections of the channel in the order of size.

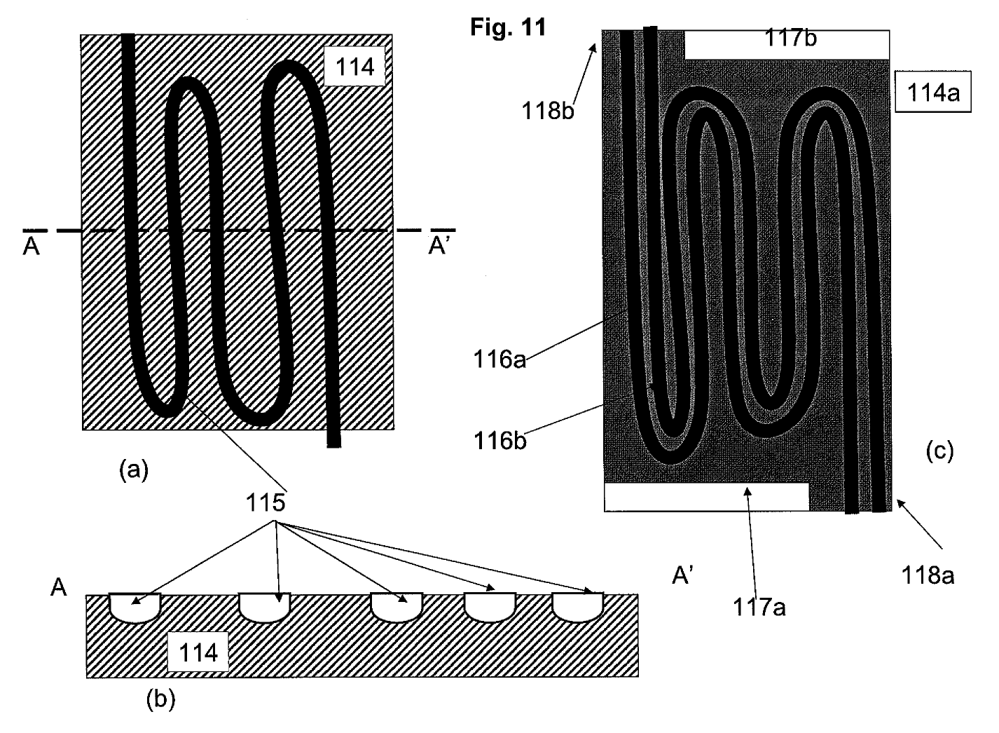

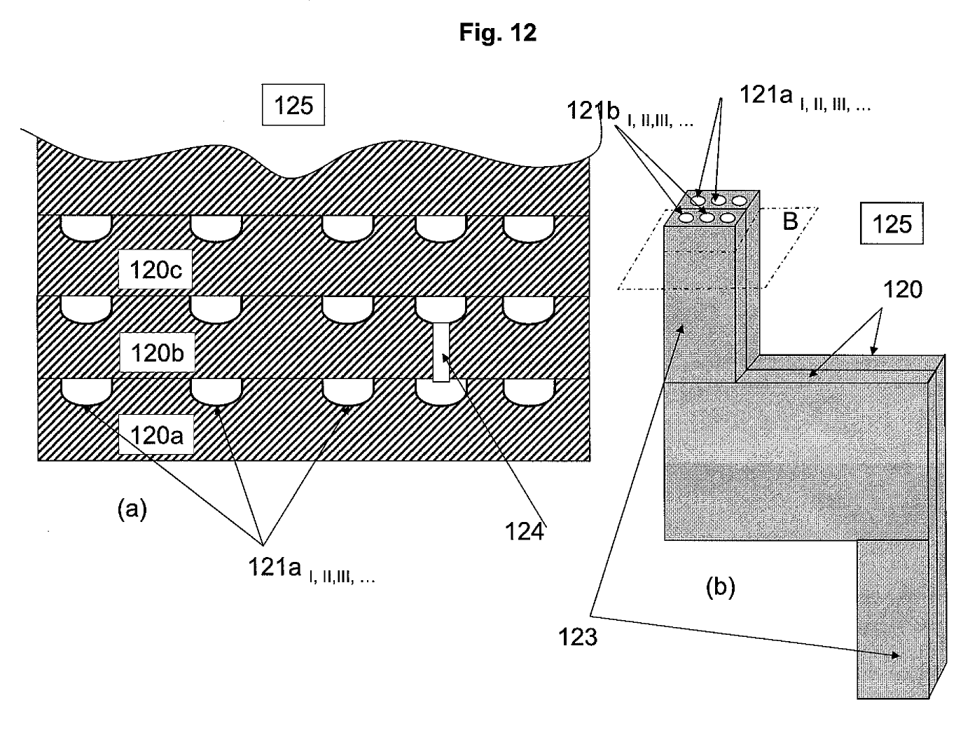

A preferred embodiment of such channel configuration is illustrated in FIG. 11. Plate 114 contains channel 115 of a curved length throughout the extents of the substrate. FIG. 11a shows an example of the curved shape of the channel in the substrate, while FIG. 11b shows a 90 degree cross section AA′ of the substrate with different sections of the channel exposed. The channels can be etched in the substrate/wafer/plate, or can be cast, or made by any other known methods. For clarity of explanation the channels are shown as zigzags to resemble a result obtained by etched cavity fabrication. Other geometries, such as semicircles, circles, rectangles, triangles and more, may be desirable and are also possible to fabricate. Furthermore, as shown in FIG. 11c, one or more channels 116a, 116b, etc. can be fabricated in a single substrate. These channels can be parallel or not to each other, of same or different lengths, with same or different cross sections, and in general can be similar or complete different in any quality. The channels can be entirely embedded in the substrate, or only part of them is fabricated in a single substrate plate, while additional plates may be required to make entire channels (for example, see connection 124 in FIG. 12a.) As shown in FIG. 11c each plate can be cut or fabricated in such a way that a recession 117a, 117b exists as to form one or more protrusions 118a, 118b. This configuration can be used for connecting the channel ends to other parts of the apparatus such as gates, fluids, electrodes, optical connections etc.

In a preferred embodiment several plates 114 can be combined via bonding, gluing or any other method known to those skilled in the art. Such preferred embodiment is schematically illustrated in FIG. 12. Several plates 120 designated in the figures as 120a, 120b, 120c, etc. of any type described previously, are stacked one on top of another. The plates can be equal or each one can have a different configuration. The plates can be aligned each on top of the other, or need not be aligned. Each plate contains one or more channels 121a_{I, II, III}, . . . , 121b_{I, II, III}, . . . , with same or different configurations. FIG. 12a schematically illustrates a cross section of stack 125 through plane B of FIG. 12b. FIG. 12b shows protrusions 123, formed from the stacking of the plates, which are combined with protrusions 118a/118b of FIG. 11c. These protrusions can serve, for example, for placing the gates of this disclosure, or as connections for fluid attachments, placement of electrodes, and/or optical sections. The channels can be made in a single plate, or as a combination of channels in different plates, for example by via channel element 124 in FIG. 12a.

The channels can be made using a variety of different techniques. Among others the channels can be made by glass fiber pulling, or composed of glass fiber bundles, and/or the channels can be made by etching the channels in Si, glass or other material wafers by known techniques. In each wafer a single channel can be made, or many channels for one or many locations can be fabricated. The wafers can be stacked and bonded using adhesives or by anodic or thermal bonding as to form an array of channels. The channels can have a complete functional length in one wafer, or the length can span between many wafers and connected through appropriate via channels.

A variety of methods can be applied to identify the sequenced segments or fluorescent-labeled sequenced fragments as they arrive to the terminal sections of the channel. As a preferred embodiment a fluorescence excitation/emission method is utilized for DNA fragment identification purposes. In the embodiment excitation light impinges at a section of a channel. This section is transparent to the wavelengths used for excitation and the resulting fluorescence emission. The excitation light source can be, but is not limited to, a laser light source. The light excitation causes fluorescence emission from the fluorescently-labeled sequenced fragments. The fluorescence wavelengths, intensities and durations are registered and stored for further processing. Fluorescence can be detected using a photo-detector arranged as a single detector or arrayed detector of any known kind appropriated to sense the required wavelengths of the fluorescence emission. For example, the sensor can be a CCD imaging element as normally used in digital photography or video recording or other imaging applications. In such case a single sensor element can be designated for a single channel many sensors can be assigned to a single channel, a single section in the channel, or many sections in a channel. In a preferred embodiment many sensors can be assigned to sense the fluorescently-labeled sequenced fragments from a section, or many sections of a single channel. These sensors can be equal, or of different kinds so that each or several of them are sensitive to different characteristics of the emanating light.

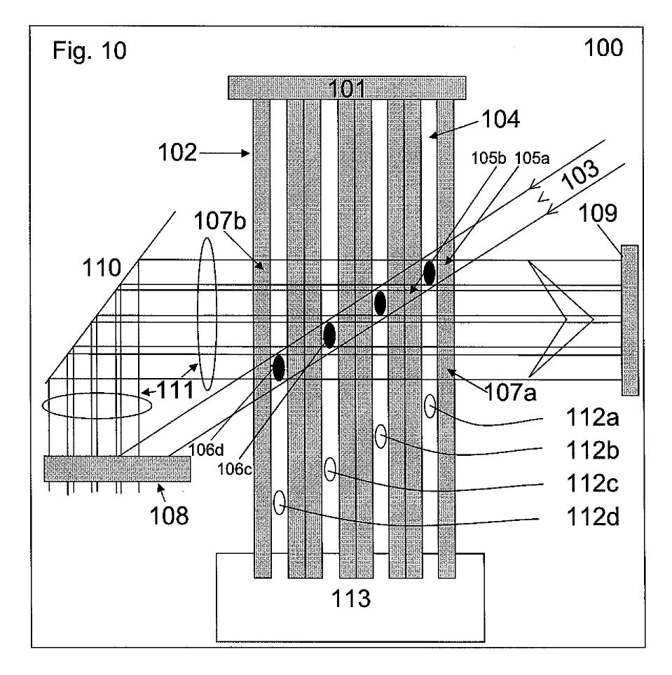

When light impinges on large bundles of channels and the fluorescence emission signal needs to be collected, the light path might be blocked, partially blocked or altered by other channels in the bundle. To resolve the interferences the optical-electronic system of the sequencing apparatus may comprise an optical detection system presented herein. The optical detection system is described in relation to FIG. 10. Other elements of the sequencing apparatus, such as gates, reservoirs and other components disclosed earlier are indicated in the figure collectively as plate 101. The channels are illustrated as a bundle 102 in a preferred embodiment of the system 100. The incident light beam 103 strikes the bundle at a non-zero angle to the bundle's axis. Each channel core is shown filled with gel 104. In the shown configuration a different section 105 of each channel is exposed to the incident light. Each different section is at a different position relative to the length of the channel axis (105a, 105b, 105c, . . . ). The fluorescence light 106 emitted at each section has a different optical path through the bundle. In particular, the fluorescence light 106 emanates from section 107 not exposed to the inducing beam and thus not blocked or not interfering with the readings from adjacent channels. The fluorescent light is collected by sensors 108, 109 as described previously. This light can be collected from many positions (108/109) relative to the bundle and from several positions at once. The sensor, or sensor array, can rely on optical elements, such as mirrors 110 and lenses 111, to direct the emission light to the senor imaging system as may be needed. The sections from where the fluorescence light is emitted are at different positions 106a, 106b, 106c, . . . In applications where the times of migration of different DNA fragments should be similar, placing electrophoresis electrodes 112 at different distances relative to the channel lengths permits the electrophoresis velocity to vary as to compensate for the interrogation position differences. The compensation can also be achieved during the data processing stage as to take into consideration the differences in traveled path lengths. DNA fragments could or are collected in an output reservoir 113 upon exiting the channels.

What is claimed is:

1. A DNA sequencing method comprising:
performing random digestion of a stretch of DNA;
performing adaptor ligation of adaptor DNA fragments to DNA segments produced in random digestion, each said adaptor DNA fragment containing a sequence which is complementary to a single DNA primer;
performing PCR amplification of the ligated segments produced in adaptor ligation, said PCR amplification utilizing said single DNA primer;
distributing the ligated segments into one or more pre-defined isolated locations of a sequencing apparatus, each said location containing DNA fragments placed thereat for capturing a unique kind of digested DNA segments;
capturing at each said location a unique kind of amplified DNA segments by hybridizing said DNA segments with said DNA fragments;
dislodging captured DNA segments from said DNA fragments at said locations;
adding DNA sequencing reaction components into said locations of the sequencing apparatus;
performing sequencing reactions at said locations of the sequencing apparatus;
performing separation of the products of the sequencing reactions in the sequencing apparatus, DNA dislodged at an individual said location being individually separated; and
determining the sequences of DNA segments captured at individual locations of the sequencing apparatus.

2. The method of claim 1 further comprising assembling the entire sequence of the original stretch of DNA using segment sequences of said stretch.

3. The method of claim 1 further comprising removing unbound DNA from said locations of the sequencing apparatus after capturing the amplified DNA segments.

4. The method of claim 3 further comprising repeating said DNA capturing followed by said unbound DNA removing one or more times.

5. The method of claim 1 wherein said random digestion is performed using restriction enzymes.

6. The method of claim 1 wherein said separation of the products of the sequencing reactions is performed by electrophoresis.

7. The method of claim 1 wherein said DNA capturing, said DNA dislodging, said sequencing reactions, said DNA separation, or said determining of DNA sequences are performed for two or more said locations in parallel.

8. A DNA sequencing apparatus comprising:
means for receiving a DNA solution, said DNA receiving means containing DNA fragments placed therein for capturing a unique kind of DNA segments from said DNA solution, and being capable of supporting a PCR reaction, a DNA sequencing reaction, single stranded DNA hybridization into double stranded DNA, or double stranded DNA strand separation;
means for performing DNA separation according to size, said DNA separation means being associated with said DNA receiving means;
means for controlling the access of DNA into said DNA receiving means and into said DNA separation means;
means for inducing fluorescence excitation in, and for detecting fluorescence emission from DNA separated in said DNA separation means; and
a computer system receiving fluorescence emission information from said fluorescence/emission means and processing said information to produce DNA sequence data.

9. A DNA sequencing apparatus comprising:
one or more isolated locations, each location comprising a reservoir which contains DNA fragments placed therein for capturing a unique kind of DNA segments from a DNA solution after dispensing the DNA solution into said reservoir, said reservoir being capable of supporting a PCR reaction, a DNA sequencing reaction, single stranded DNA hybridization into double stranded DNA, or double stranded DNA strand separation;
one or more channels performing DNA separation according to size, said channels being associated with one or more of said reservoirs;
an optical system which induces fluorescence excitation in, and detects fluorescence emission from separated DNA migrating in said channels; and
a computer system receiving fluorescence emission information from said optical system and processing said information to produce DNA sequence data.

10. The apparatus of claim 9 further comprising one or more gates controlling the access of substances into said reservoirs and into said channels.

11. The apparatus of claim 10 wherein said DNA fragments are attached to a surface of said gates.

12. The apparatus of claim 11 wherein said gates comprise a DNA holding structure which places said DNA fragments into one or more said reservoirs in parallel and opens or closes all the locations at the same time.

13. The apparatus of clam 10 wherein said gates comprise at least one translating element, said translating element moving between a first position, which allows said DNA segment capture with said DNA fragments in a reservoir, and a second position which allows for a sequencing reaction to proceed in a reservoir.

14. The apparatus of claim 13 wherein said translating element has a substantially spherical shape.

15. The apparatus of claim 13 or 14 wherein said DNA fragments are attached to said translating element.

16. The apparatus of claim 9 or 10 further comprising a DNA holding structure which places said DNA fragments into one or more said reservoirs.

17. The apparatus of claim 10 wherein said gates comprise a rotating element, said rotating element swiveling between a first position, which allows said DNA segment capture with said DNA fragments in a reservoir, and a second position with allows for a sequencing reaction to proceed in a reservoir.

18. The apparatus of claim 17 wherein said DNA fragments are attached to said rotating element.

19. The apparatus of claim 18 wherein said rotating element in the first position places said DNA fragments into a reservoir and in the second position removes said DNA fragments from the reservoir while closing another reservoir.

20. The apparatus of claim 10 wherein said gates comprise a bending element, said bending element flexing between a first position, which allows said DNA segment capture with said DNA fragments in a reservoir, and a second position with allows for a sequencing reaction to proceed in a reservoir.

21. The apparatus of claim 10 further comprising means for actuating said gates based on electrostatic, electromagnetic, piezoelectricity or thermal principle.

22. The apparatus of claim 10 in which said channels are hollow fiber bundles.

23. The apparatus of claim 10 in which one or more of said channels are defined into one or more substrate plates.

24. The apparatus of claim 23 in which the substrate is glass or silicone or polymers.

25. The apparatus of claim 23 in which a single channel spans more than one substrate plate.

26. The apparatus of claim 24, 25 or 26 in which said channels form curved patterns as to increase their length per unit of substrate area.

27. The apparatus of claim 9 or 10 in which said optical system detects fluorescence emission from more than one channel at the same time while detecting said fluorescence individually for each channel.
